(19) United States
(12) Patent Application Publication
(10) Pub. No.: US 2024/0033027 A1
(43) Pub. Date: Feb. 1, 2024

(54) IN-SITU FENESTRATION DEVICES WITH MAGNETIC LOCATORS AND HEATING ELEMENTS

(71) Applicant: Medtronic Vascular, Inc., Santa Rosa, CA (US)

(72) Inventors: Keith D. PERKINS, Santa Rosa, CA (US); Mark L. STIGER, Santa Rosa, CA (US); Jason S. BOWE, Blaine, MN (US); Woong KIM, Blaine, MN (US); Nathan B. WIEMEYER, Santa Rosa, CA (US)

(21) Appl. No.: 18/212,540

(22) Filed: Jun. 21, 2023

Related U.S. Application Data
(60) Provisional application No. 63/393,039, filed on Jul. 28, 2022.

(51) Int. Cl. A61B 34/00; A61B 18/14; A61F 2/07

(52) U.S. Cl. CPC A61B 34/73; A61B 18/1492; A61F 2/07; A61B 2034/731; A61B 2018/00601

(57) ABSTRACT

An in-situ fenestration device for locating a fenestration site and for forming a fenestration. The in-situ fenestration device includes first and second magnetic locators configured to magnetically mate in a mated position within a vasculature of a patient at the fenestration site. The in-situ fenestration device also includes first and second heating elements configured to heat the first and second magnetic locators to form the fenestration at the fenestration site.

### CROSS-REFERENCE TO RELATED APPLICATION

This application claims the benefit of U.S. provisional application Ser. No. 63/393,039, filed Jul. 28, 2022, the disclosure of which is hereby incorporated in its entirety by reference.

### TECHNICAL FIELD

The present disclosure relates to in-situ fenestration devices with magnetic locators and heating elements.

### SUMMARY

In a first embodiment, an in-situ fenestration device for locating a fenestration site and for forming a fenestration is disclosed. The in-situ fenestration device includes first and second magnetic locators configured to magnetically mate in a mated position within a vasculature of a patient at the fenestration site. The in-situ fenestration device also includes first and second heating elements configured to heat the first and second magnetic locators to form the fenestration at the fenestration site.

In a second embodiment, an in-situ fenestration device for locating a fenestration site and for forming a fenestration is disclosed. The in-situ fenestration device includes a catheter having a bendable distal portion, a trench, and a magnetic device carrying a first magnetic locator. The bendable distal portion is configured to bend such that the magnetic device transitions from a delivery position to a deployment position through the trench and about a pivot axis. The in-situ fenestration device also includes a second magnetic locator. The first and second magnetic locators are configured to magnetically mate in a mated position within a vasculature of a patient at the fenestration site. The in-situ fenestration device further includes first and second heating elements configured to heat the first and second magnetic locators to form the fenestration at the fenestration site.

In a third embodiment, an in-situ fenestration device for locating a fenestration site and for forming a fenestration is disclosed. The in-situ fenestration device includes first and second magnetic locators configured to magnetically mate in a mated position within a vasculature of a patient at the fenestration site. The first and second magnetic locators have first and second complimentary shapes, respectively, configured to align the first and second magnetic locators to magnetically mate in the mated position. The in-situ fenestration device further includes first and second heating elements configured to heat the first and second magnetic locators to form the fenestration at the fenestration site.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIG. 2A depicts a partial cut away, perspective view of an abdominal aorta and a balloon protection and location device extending within the abdominal aorta and including spherical balloons.

FIG. 2B depicts a partial cut away, perspective view of an abdominal aorta and a balloon protection and location device extending within the abdominal aorta and including crescent moon shaped balloons.

FIG. 2C depicts a partial cut away, perspective view of a balloon protection and location device having balloons in an inflated state and another balloon independently in a deflated state.

FIG. 2D depicts a schematic view of a deployment of a balloon protection and location device being deployed within the abdominal aorta of a patient.

FIG. 3A depicts a partial cut away, perspective view of a deployment of an in-situ fenestration device at a covered region of a stent graft covering an ostium of a renal artery.

FIG. 3B depicts a partial cut away, perspective view of the in-situ fenestration device where an inner catheter has a heated tip configured to create an aperture in the wall of a stent graft.

FIG. 3C depicts a partial cut away, perspective view of independent deflation of a balloon to form a space to accommodate the heated tip and balloon of the inner catheter of the in-situ fenestration device.

FIG. 3D depicts a partial cut away, perspective view of balloon of the inner catheter of the in-situ fenestration device in an inflated state.

FIG. 3E depicts a partial cut away, perspective view of the retraction of the balloon of the inner catheter into a retracted state, thereby pulling the graft material adjacent the aperture into a conical section of the outer sheath.

FIG. 3F is a cross section, side view of the distal region of the outer sheath including the conical section having a series of circumferential heating elements.

FIG. 3G depicts a magnified, partial cut away, perspective view of a cutting step for cutting the inwardly folded graft material with one of the circumferential heating elements.

FIG. 3H depicts a magnified, perspective view of the outer sheath including spaced apart circumferential radiopaque (RO) markers located on the outer surface of the outer sheath.

FIGS. 4A through 4E depict a steerable catheter configured to gain access to a branch blood vessel by locating the branch blood vessel and positioning the steerable catheter at the location.

FIGS. 5A through 5J depict various devices configured to grasp graft material of a deployed stent graft at a fenestration site.

FIGS. 6A through 6D depict an RF ablation device configured to cut graft material of a deployed stent graft at a fenestration site and remove the cut graft material from the fenestration site.

FIGS. 7A through 7G depicts mechanical cutting devices configured to cut graft material at a fenestration site.

FIGS. 8A through 8D depict an embodiment directed to locating, gripping, cutting, and removing of graft material at a fenestration site.

FIGS. 9A through 9E depict embodiments directed to locating, gripping, cutting, and removing of graft material at a fenestration site.

FIGS. 10A though 10E depict embodiments directed to gripping and cutting of graft material at a fenestration site.

FIG. 11 depicts a schematic, side view of a magnetic locator system configured to locate fenestration site on a deployed stent graft.

FIGS. 12A through 12F depict schematic, side views of distal bendable portions and catheters of magnetic locator devices.

FIGS. 13A through 13D depict images of a magnetic locator device configured to be deployed within a patient's vasculature using the femoral/iliac arteries.

FIGS. 14A and 14B depict schematic views of embodiments of mechanisms for magnetizing magnetic locator devices.

FIGS. 14C and 14D depict schematic views of the mating of first and second magnetic locator devices and heating the magnetic components using inductive coils.

FIG. 15 depicts a side view of a steerable catheter device.

FIGS. 16A and 16B depict schematic views of holes formed using a magnetic locator system of one or more embodiments.

FIGS. 17A through 17F depict schematic views of alternative embodiments of grommet stents and expanding flanges on either side of the graft wall to strengthen the edge of the aperture formed by the magnetic locator system.

FIG. 18A depicts a schematic side view of an abdominal aorta and the use of a locator catheter to locate a fenestration site.

FIG. 18B depicts a schematic side view of an alternative use of a magnetized thermocouple to locate a fenestration site and to form a fenestration.

FIGS. 19A and 19B depict schematic, side views of a mechanism to size a fenestration using variably sized magnetized thermocouples.

FIG. 20 depicts a schematic side view of an aortic arch and the use of a small profile catheter to locate a fenestration site therein.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

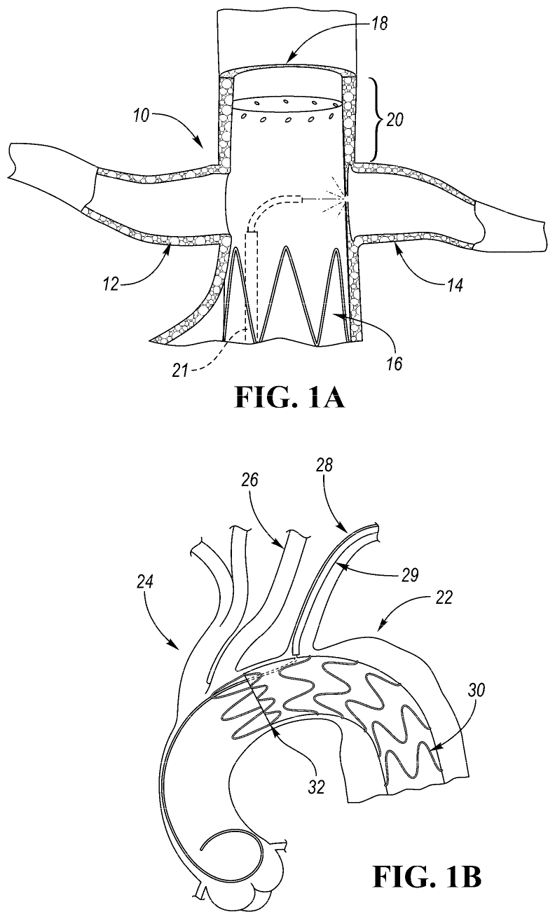

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching cannulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Eye cautery (e.g., thermal energy) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

(Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel cannulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one embodiment, a balloon protection and location device is disclosed. The balloon protection and location device includes a catheter having a distal region and a number of balloons secured to the distal region of the catheter. Each of the balloons may include a marker configured to mark a location of the respective balloon relative to an ostium of a blood vessel. One or more of the balloons may be configured to protect a vasculature of a patient from an in-situ fenestration of a region of a stent graft located in or around the ostium of the blood vessel.

In another embodiment, an in-situ fenestration device for forming a fenestration in a stent graft is disclosed. The device includes an outer sheath and an inner catheter translatable relative to and disposed within the outer sheath. The inner catheter includes a heated tip configured to form an initial aperture in the graft material of the stent graft when the inner catheter is translated from a retracted position to a deployed position. The inner aperture is configured to permit the inner catheter to extend through the graft material.

The inner catheter includes a balloon configured to inflate into an inflated state to capture adjacent graft material adjacent to the aperture when the inner catheter is translated from the deployed state to the retracted state. The outer sheath includes an inner cavity including heating elements configured to form a fenestration in the graft material and to selectively cauterize a perimeter portion of the graft material around the fenestration.

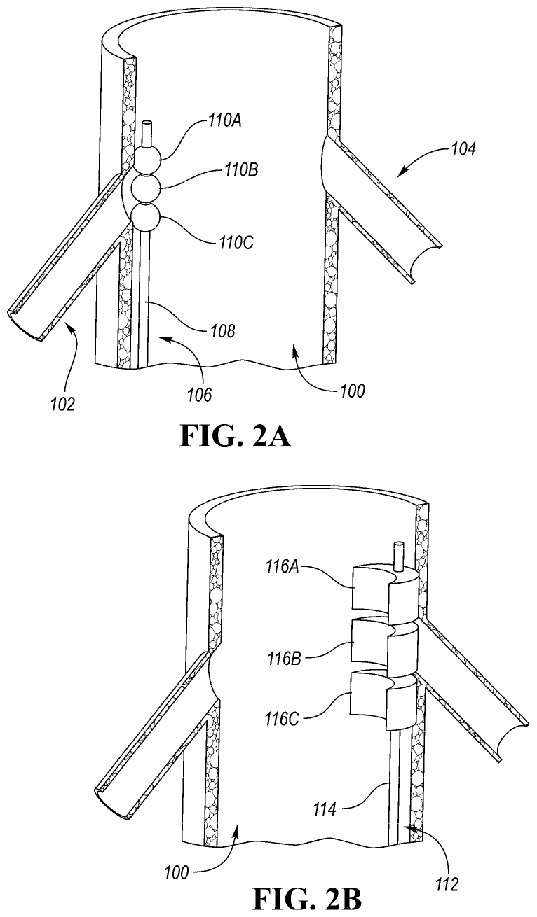

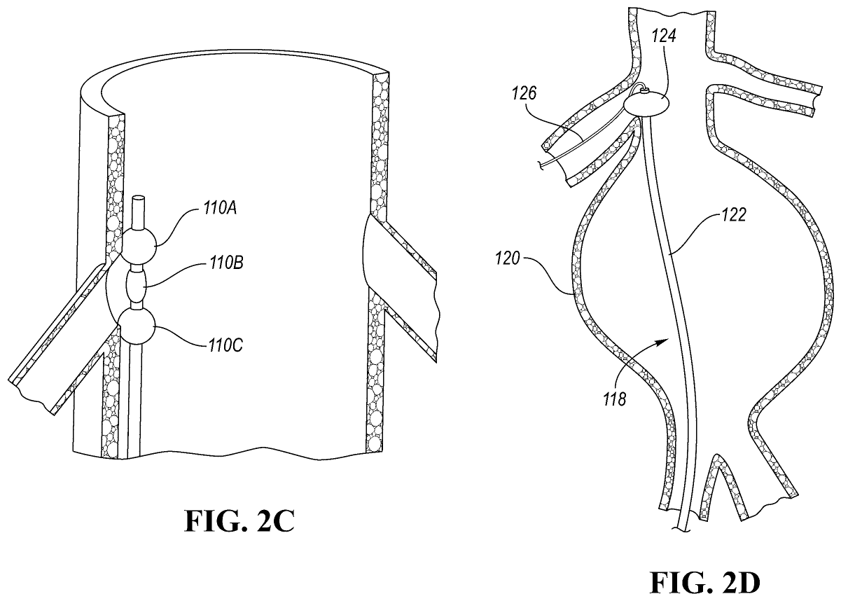

One or more embodiments disclose a balloon protection and location device. FIG. 2A depicts a partial cut away, perspective view of abdominal aorta 100 having right renal artery 102 and left renal artery 104 extending therefrom and balloon protection and location device 106 extending within abdominal aorta 100. Balloon protection and location device 106 may be used with the renal arteries and other blood vessels to locate an ostium of the blood vessel and to protect the blood vessel during a subsequent in-situ fenestration of a region of a stent graft located about the ostium of the blood vessel. Balloon protection and location device may be used with any in-situ fenestration device, including those disclosed in the present application or other fenestration devices (e.g., a laser fenestration device). The balloon protection and location device may also be used to help visualize the location of the balloons relative to the ostium of a blood vessel.

Balloon protection and location device 106 includes catheter 108 and balloons 110A, 110B, and 110C spaced apart from each other and secured to a distal region of catheter 108. As shown in FIG. 2A, balloons 110A, 110B, and 110C are spherical, but in other embodiments, the balloons may take on a different shape (e.g., a cylindrical shape). As shown in FIG. 2A, balloon protection and location device 106 has three (3) balloons. Depending on the intended peripheral blood vessel for using balloon protection and location device 106, there can be less or more balloons affixed to the catheter (e.g., 1, 2, 4, 5, 6, 7, and 8 balloons or any range therein). Balloons 110A, 110B, and 110C may be compliant balloons such that the balloons at least partially comply (e.g., deform) with the anatomy within the vasculature of the patient.

Balloons 110A, 110B, and 110C may have a diameter corresponding to the blood vessels in which balloon protection and location device 106 is used. Balloons 110A, 110B, and 110C may have a diameter or a range of diameters of any two of the following: 6.4, 6.5, 6.6, 6.7, 6.8, 6.9, 7.0, 7.1, and 7.2 millimeters (e.g., when balloon protection and location device 106 is used with renal arteries.) The spacing between balloons 110A, 110B, and 110C may be any of the following values or in a range of any two of the following values: 0.7, 0.8, 0.9, 1.0, 1.1, and 1.2 millimeters.

FIG. 2B depicts a partial cut away, perspective view of abdominal aorta 100 and a balloon protection and location device 112 extending within abdominal aorta 100. Balloon protection and location device 112 includes catheter 114 and balloons 116A, 116B, and 116C. As shown in FIG. 2B, balloons 116A, 116B, and 116C are crescent moon shaped, but in other embodiments, the balloons may take a different shape (e.g., cylindrical or spherical shaped balloons). Balloons 116A, 116B, and 116C may be noncompliant balloons such that when the balloons are at a relatively high pressure the balloons do not comply with the anatomy within the vasculature of the patient. In one or more embodiments, the balloons shown in FIGS. 2A and 2B may be mixed and matched to form a set of balloons with two or more balloon types depending on the application of the balloon protection and location device.

Balloons 110A, 110B, and 110C (or 116A, 116B, and 116C) may be independently inflated and deflated (e.g., before or after deployment) to aid in locating the ostium of a blood vessel and provide added control to a clinician. FIG. 2C depicts balloon protection and location device 106 having balloons 110A and 110C in an inflated state and balloon 110B in a deflated state. Balloons 110A, 110B, and 110C may be independently inflated with a contrast material supplied from the lumen of catheter 108. Balloons 110A, 110B, and 110C may include radio opaque (RO) markers for visibility within the vasculature of the patient. The balloon(s) may also have a protective coating to protect the balloon(s) from applied heat. The balloon(s) may be partially filed with a solution (e.g., saline solution) to dissipate heat (e.g., heat generated from the in-situ fenestration process). The balloon(s) may have any combination of the above properties and the inflation material may have a combination of the disclosed properties (e.g., contrast and heat dissipation).

FIG. 2D depicts a schematic view of a deployment of balloon protection and location device 118 being deployed within abdominal aorta 120 of a patient. FIG. 2D generally depicts catheter 122 and balloons 124 (not shown individually in the figure) situated at the distal end of balloon protection and location device 118. A visceral artery (e.g., a renal artery as shown in FIG. 2D) can be located and accessed using guidewire 126. Catheter 122 may be advanced along guidewire 126 to an intended position (e.g., an alignment between one or more balloons and the ostium of the visceral artery). During deployment, the balloons 124 may be at least partially or completely deflated to make travel easier within the patient's vasculature. Once the balloons are in the intended position (potentially aided by the contrast material and/or RO markers), one or more of the balloons (e.g., independently or in parallel) may be inflated to cover the visceral vessel ostia to protect it during in-situ fenestration.

The balloon protection and location device may have one or more benefits. The device may provide guidance for creating a fenestration at or near the branch ostium. Depending on the type, inflation, and/or delivery, the balloons of one or more embodiments may provide an anchoring benefit at a branch vessel to reduce or minimize relative motion between the anatomy and location device, without completely occluding the ostium. The balloons of the device may protect vasculature from damage. The balloon protection and location device may be used with current fenestration technology (e.g., laser or RF), thereby providing options to clinicians, or it may be used with any fenestration technology disclosed herein. The device may allow perfusion of visceral arteries during the procedure, thereby minimizing trauma to branch organs.

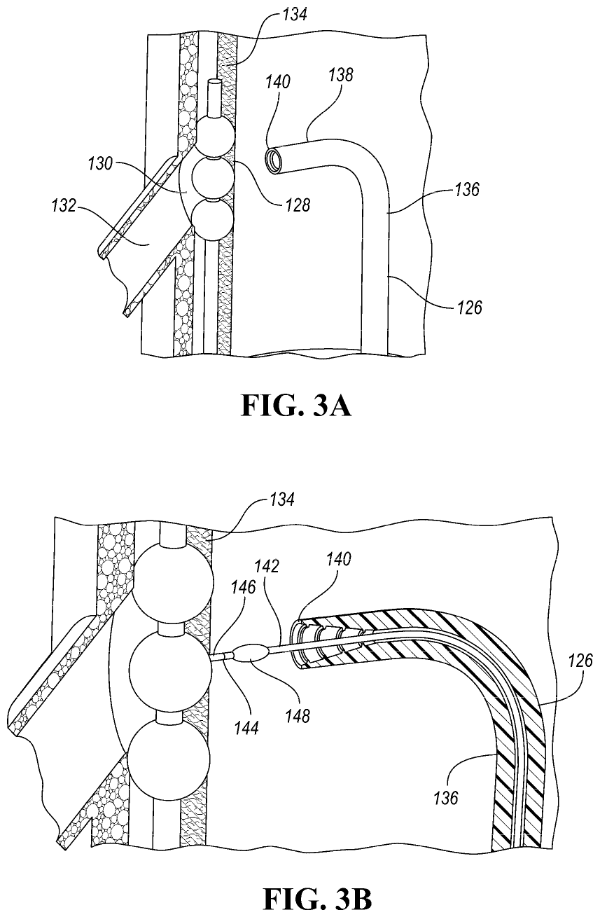

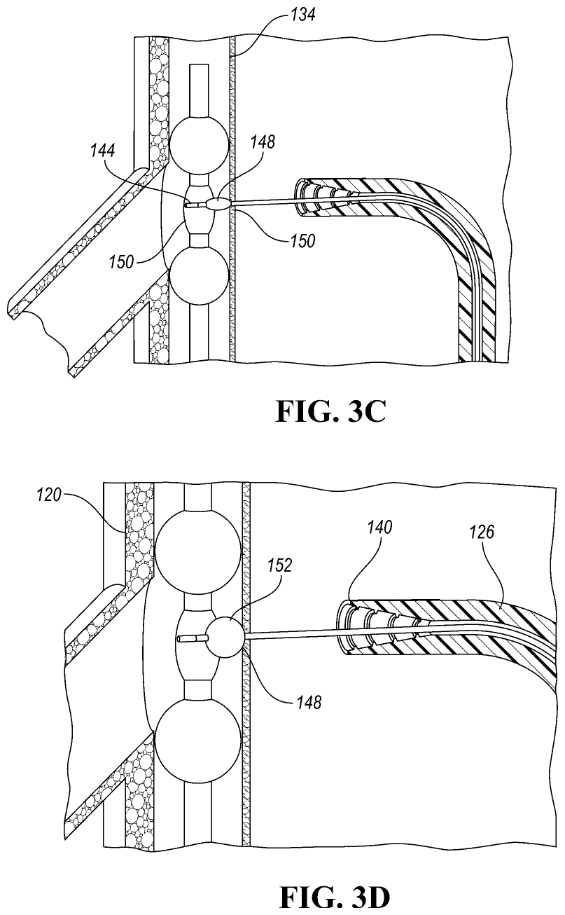

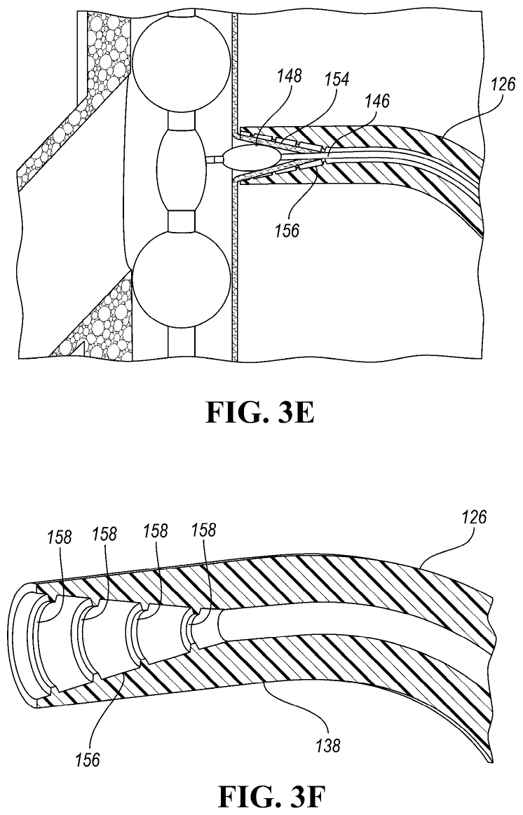

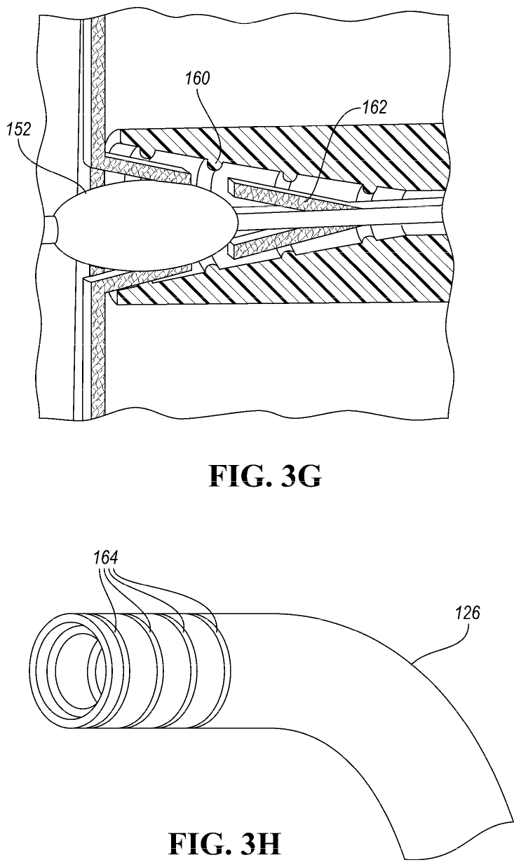

FIG. 3A depicts a partial cut away, perspective view of a deployment of an in-situ fenestration device 126 at covered region 128 covering ostium 130 of renal artery 132. As shown in FIG. 3A, stent graft 134 is deployed at an intended position, thereby creating covered region 128 covering ostium 130 of renal artery 132. The stent graft 134 may be covering or blocking the renal artery 132 due to a hostile anatomy below the renal arteries, as described above. Stent graft 134 is deployed in a radially expanded state after balloon protection and location device 126 is deployed at its intended position, which forms an alignment of the balloon(s) and the renal artery ostium. In the radially expanded state, stent graft 134 helps to maintain one or more of the balloons at their intended position as the balloon protection and location device 118 is sandwiched between the vessel wall and the stent graft 134. Once stent graft 134 is deployed, outer sheath 136 of device 126 is tracked toward renal artery 132 (e.g., independently, over inner catheter 142, or over another guidewire). Once outer catheter 136 is in the vicinity of renal artery 132, which is an intended location, distal region 138 of outer sheath 136 may be bent a number of degrees (e.g., about 90 degrees as shown in FIG. 3A) to align open distal end 140 of outer sheath 136 with one or more of the balloons and the renal artery ostium. The outer sheath can be a steerable sheath. The outer catheter may have one or more RO markers or bands at the distal end 140 (described further, below) which may be aligned with RO markers of the balloon protection and location device 126 or contrast material in the balloons thereof to ensure the intended alignment with the ostium.

FIG. 3B depicts a schematic view of in-situ fenestration device 126 where inner catheter 142 has heated tip 144 configured to create aperture 146 in the wall of stent graft 134. Inner catheter 142 is disposed through outer sheath 136 such that it extends through open distal end 140 of outer sheath 136. Balloon 148 is secured around the outer surface of inner catheter 142 and is located proximal heated tip 144. Balloon 148 may be fixedly connected to inner catheter 142. Heated tip 144 may be energized to generate heat (e.g., using thermal heat) used to create aperture 146 when advancing catheter 142 penetrates through the graft material of stent graft 134. Inner catheter 142 and/or heated tip 144 may have a diameter of any of the following values or in a range of any two of the following values: 0.5, 0.6, 0.7, 0.8, 0.9, 1.0, 1.1, 1.2, 1.3, 1.4, 1.5, 1.6, 1.7, 1.8, 1.9, and 2.0 millimeters. Heated tip 144 may be heated using any suitable technology, such as resistive heating or RF energy. Alternatively, instead of creating aperture 146 using heat, the distal tip of the inner catheter 142 may have a cutting mechanism, such as a sharp tip or an ultrasonic cutting tool, or any other suitable cutting technology.

FIG. 3C depicts a partial cut away, perspective view of independent deflation of balloon 150 to form a space for heated tip 144 and balloon 148 to pass through. Balloon 150 may be completely deflated or partially deflated. A partially deflated balloon may provide heat dissipation via the remaining inflation fluid, which may provide protection to surrounding tissue when heated tip 144 is activated. Balloon 148 may be deflated before heated tip 144 is advanced toward covered region 128. After heated tip 144 and balloon 148 pass through aperture 146 and at least partially resides in the space formed by deflated balloon 150, both heated tip 144 and balloon 148 are located outside of stent graft 134. As shown in FIG. 3C, balloon 148 is in a deflated state as it passes through aperture 146 and into the space formed by deflated balloon 150.

FIG. 3D depicts a partial cut away, perspective view of balloon 148 in inflated state 152. In inflated state 152, balloon 148 has a diameter or width that is greater than the diameter or width of aperture 148, thereby allowing graft material adjacent to aperture 146 to be drawn inward toward open distal end 140 of outer sheath 126 as further described in connection with FIG. 3E.

FIG. 3E depicts a partial cut away, perspective view of the retraction of balloon 148 into a retracted state, thereby pulling graft material 154 into conical section 156 of outer sheath 126. As shown in FIG. 3E, as balloon 148 is retracted into conical section 156, balloon 148 deforms into a deformed state (e.g., balloon 148 is a conformal balloon), thereby urging inwardly folded graft material 154 onto the surface of conical section 156. In this position, aperture 146 is situated at the apex of conical section 156.

FIG. 3F is a cross section, side view of distal region 138 of outer sheath 126 including conical section 156 having a series of circumferential heating elements 158. In the embodiment shown, there are four heating elements 158, however, there may be 2, 3, 5, or more heating elements, or any range therein. Circumferential heating elements 158 are integrated into the wall of conical section 156 and spaced apart from each other such that each circumferential heating element 158 has a different diameter where the diameters increase from the apex/proximal end of conical section 156 toward the base/distal end of conical section 156. As shown in FIG. 3F, each of circumferential heating elements 158 are equally spaced along the longitudinal axis of conical section, however, in other embodiments the heating elements may be unequally spaced. For example, the distance between heating elements could increase or decrease from one end of the conical section to the other. The angle of the conical section 156 may be larger or smaller than shown. For example, a relatively minor angle may allow better contact and conformity between the balloon and the graft material. While the inner surface is shown as having a conical section 156, in other embodiments the inner surface may be cylindrical or substantially cylindrical. In these embodiments, the heating elements may still be axially spaced as shown in FIG. 3F.

FIG. 3G depicts a magnified, partial cut away, perspective view of a cutting step for cutting inwardly folded graft material 154 with one of the circumferential heating elements 158. The size of the cut hole is congruent or related with the diameter of the circumferential heating element 158 used to make the cut. A clinician can select the cut hole size by using a selection handle (not shown). The selection handle may be configured to activate the circumferential heating element 158 corresponding to the desired cut hole diameter. In general, selecting a heating element closer to the distal creates a larger diameter fenestration, as more graft material is removed. As shown in FIG. 3G, circumferential heating element 160 is selected and activated to cut inwardly folded graft material 154 to create the cut hole and scrap graft material 162. Depending on the degree and/or uniformity of folding of graft material 154, the size of the hole may be substantially similar to the diameter/circumference of the activated heating element or it may be larger. If there is a large degree of infolding then the corresponding hole diameter may be significantly larger than the diameter of the heating element or even the outer sheath 126. Accordingly, the device may be used to create relatively large fenestrations without a correspondingly large sheet diameter. This may allow for little or no post-dilation of the fenestration, which may increase the durability and seal of the fenestration with a subsequent branch stent graft.

In one embodiment, there may be only a single heating element 158. In this embodiment, the size of the opening created by the heating element may be determined by the amount of graft material pulled into the distal end of the outer sheath. For example, if the balloon 152 is retracted only slightly, a relatively small opening may be formed, but if the balloon is retracted further then more graft material is pulled into the sheath and is subsequently removed by the heating element. This arrangement may be less consistent at forming the desired fenestration size but may allow for a simpler device design and construction.

The heat applied to circumferential heating element 158 cauterizes the fabric creating the hole, thereby reducing or eliminating frayed edges. Once the fabric hole is made, balloon 152 can be withdrawn through the lumen of outer sheath 136 to extract and capture the scrap graft material 162 along with the optional use of vacuum aspiration, thereby reducing emboli risk.

In one or more embodiments, balloon 152 can be inflated (fully or partially) within conical section 156 to reduce unwanted folding of inwardly folded graft material 154 prior to the cauterization step. In one or more embodiments, folding may be desired to create a fenestration with a relatively small device profile. The heating time and/or temperature can be optimized to reduce frayed edges of the cut fabric. As the heating elements are inside the catheter, this reduces risk of vessel trauma. The inflation fluid of the balloon 152 may serve to dissipate heat from the heating element, which may prevent the balloon from being punctured or popped by the applied heat. The inflation fluid may be selected to have a high heat absorption capacity. The balloon may be formed of a heat resistant material.

FIG. 3H depicts a magnified, perspective view of outer sheath 126 including spaced apart circumferential radiopaque (RO) markers 164 located on the outer surface of outer sheath 126. RO markers 164 may be aligned with heating elements 158 to help a clinician visualize the location of one or more of the heating elements relative to outer sheath 126 and the stent graft. In the embodiment shown, there are four RO markers, however, there may be 2, 3, 5, or more heating elements, or any range thereof. As with the heating elements, the RO markers may be equally or unequally spaced, and the distance between RO markers may increase or decrease from one end of the distal region to the other end.

The in-situ fenestration device may include one or more benefits. The device includes a mechanism to locate and communicate the branch vessel ostium. The initial penetration hole is relatively small. If the initial penetration hole is in the wrong location, the clinician can stop the rest of the procedure and allow a clot to form in the relatively small penetration hole. The various sized heating elements allow for multiple sized holes to be made, which may be large enough to reduce or eliminate the need for post-dilation. The material can be extracted once it is cut (e.g., reduce or eliminate potential emboli). The edges of the material are melted to prevent or mitigate fraying. The device may adapt to a wide range of anatomies.

In an embodiment, an in-situ fenestration device for forming a fenestration in a stent graft is disclosed. The device may include an outer sheath and an inner catheter translatable relative to and disposed within the outer sheath. The inner catheter includes a grasping device for grasping graft material at a fenestration site. The inner catheter includes a cutter (e.g., an RF heating element or mechanical cutter), which may be offset the distal tip of the outer sheath. The cutter may be configured to cut the grasped graft material inward the distal tip of the outer sheath to form cut graft material. The grasping device may be configured to remove the cut graft material from the fenestration site.

In one or more embodiments, a steerable catheter configured to locate, access, and perform an in-situ fenestration at a fenestration site is disclosed. The in-situ fenestration may be performed using a radio frequency (RF) ablation energy source. One or more embodiments use a visualization technique via inner graft tracking with a steerable catheter system to access a branch blood vessel (e.g., peripheral blood vessel). Graft material removal may be performed using an internally located RF energy ring contained within the catheter tip. Graft frame and/or tissue contact may be reduced or eliminated using an RF energy ring contained within the catheter tip. The steerable catheter system may use an RF pulse synchronized with a vacuum to aspirate graft material and to vaporize emboli for removal from the patient's vasculature. Emboli can be anything foreign that tracks down stream a blood vessel such as air or foreign material.

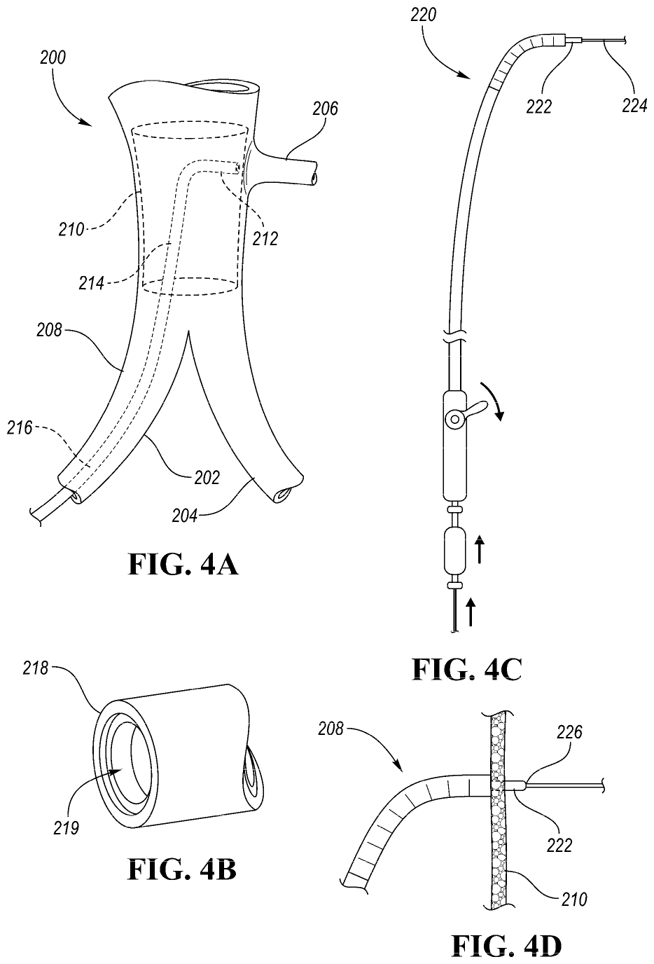

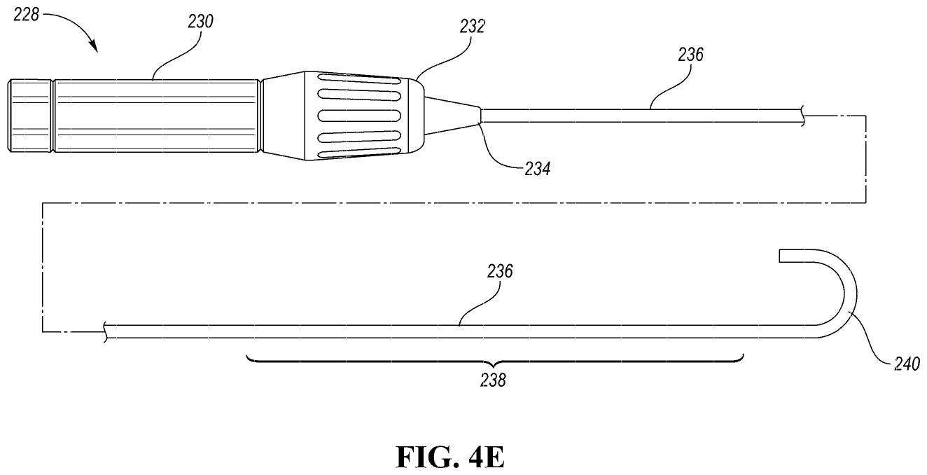

FIGS. 4A through 4E depict a steerable catheter configured to gain access to a branch blood vessel by locating the branch blood vessel and positioning the steerable catheter at the location. FIG. 4A depicts a schematic side view of abdominal aorta 200 and right common iliac 202 and left common iliac 204 extending therefrom, and steerable catheter 208 tracking through right common iliac 202. Abdominal aorta 200 also has peripheral artery 206 (e.g., a renal artery) extending therefrom. Steerable catheter 208 tracks from right common iliac 202 into abdominal aorta 200 and into implanted stent graft 210. Steerable catheter 208 includes distal primary flex region 212 and secondary bending region 214 to collectively provide support and apposition to the graft material of implanted stent graft 210. The stent graft 210 may be covering or blocking the peripheral artery 206 due to a hostile anatomy below the peripheral artery 206 (e.g., renal artery as described herein). Steerable catheter 208 includes lumen 216 defined by steerable catheter 208. A vacuum may be applied to lumen 216 to grasp the graft material of stent graft 210 and/or to assist in removing graft material of stent graft 210 as disclosed herein. Steerable catheter 208 also includes radio frequency (RF) ablation component 219 (e.g., an RF ablation ring), which can be disposed internal to distal tip 218 of steerable catheter 208, as shown in FIG. 4B. While component 219 is described in this embodiment as an RF ablation element, it may also be any suitable energy-based cutting mechanism, such as a resistive heating element.

FIG. 4C depicts a schematic view of steerable catheter deployment device 220 configured to deploy steerable catheter 208, needle 222 (e.g., hollow needle) through steerable catheter 208, and guidewire 224 through needle 222. Once steerable catheter is aligned with peripheral artery 206, needle 222 is deployed through steerable catheter 208.

FIG. 4D depicts needle 222 piercing graft material of stent graft 210 by advancing needle 222 into the graft material to form an aperture in the graft material. Once needle 222 is advanced past the graft material, guidewire 224 is tracked through needle 222 of steerable catheter 208, thereby advancing past distal tip 226 of needle 222 and into peripheral artery 206.

FIG. 4E depicts a side view of steerable catheter deployment device 228 configured to deploy a steerable catheter into a vasculature of a patient. Catheter deployment device 228 includes handle 230 configured to be grasped by a clinician when deploying the steerable catheter. Catheter deployment device 228 includes knob 232 configured to steer shaft 236 within the patient's vasculature and to adjust the position of shaft 236 and the orientation of a distal portion of steerable catheter deployment device 228. Shaft 236 includes primary flexible section 240 and secondary flexible section 238, which collectively provide support and apposition to the graft material of an implanted stent graft. For example, rotation of knob 232 may cause section 240 to bend from a straight configuration to a curved configuration (as shown). The degree of rotation of knob 232 may control the degree of curvature in section 240. In at least one embodiment, section 240 may be bent at a 90 degree angle, or substantially thereabout, in order to engage the wall of the stent graft in a perpendicular configuration. A non-limiting example of a steerable catheter device is the Heli-FX steerable sheath available from Medtronic PLC of Minneapolis, Minnesota.

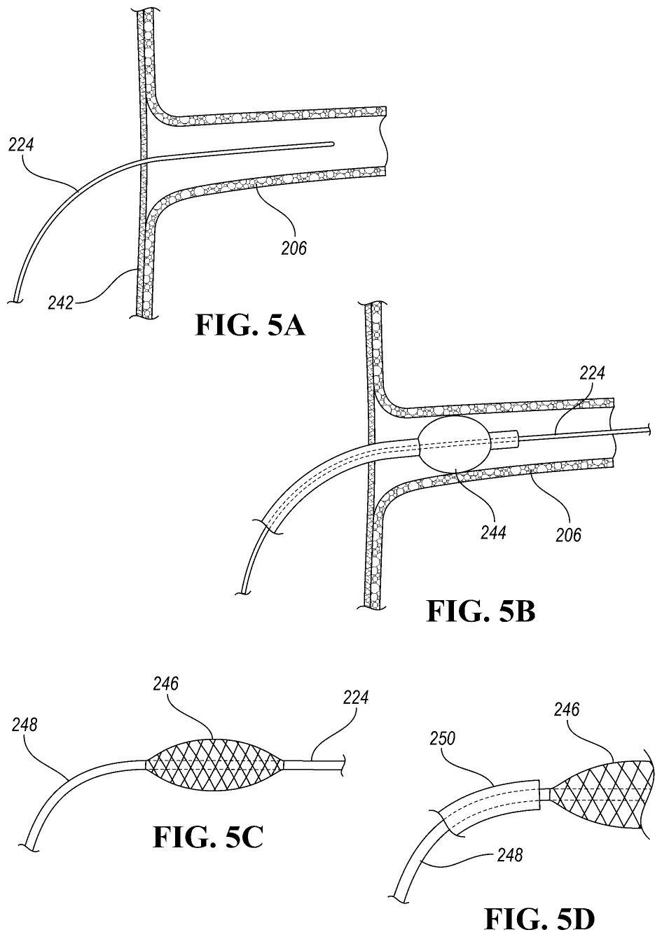

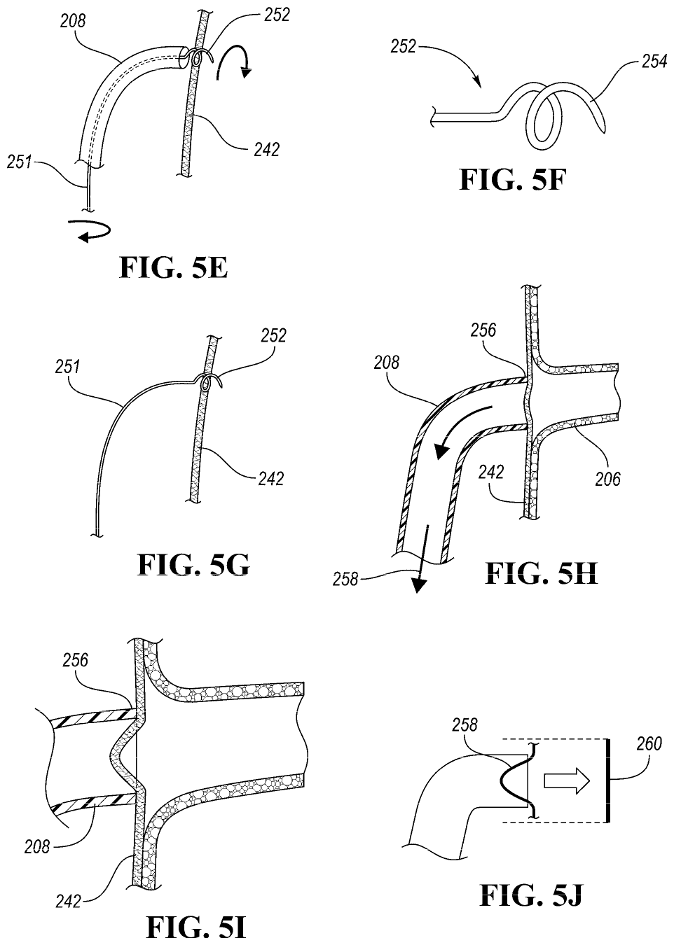

FIGS. 5A through 5J depict various devices configured to grasp graft material of a deployed stent graft at a fenestration site. FIG. 5A depicts a schematic view of the placement of guidewire 224 beyond graft material 242 of an implanted stent graft. Guidewire 224 extends through the aperture formed by advancing needle 222 through the graft material. As shown in FIG. 5A, guidewire 224 is partially located within peripheral artery 206. FIG. 5B depicts a schematic view of the advancement of a catheter (e.g., steerable catheter 208 or an inner catheter tracked through steerable catheter 208) over guidewire 224 where balloon 244 is secured to the catheter. As shown in FIG. 5B, balloon 244 is in an inflated state within peripheral artery 206. Balloon 244 in a deflated state (not shown in FIG. 5B) is passed through the aperture formed by needle 222 along with the catheter. Once balloon 244 in the deflated state passes through graft material 242, balloon 244 may be inflated with a fluid (e.g., a liquid or a gas) delivered through the catheter. Balloon 244 may then be retracted to a grasping position where balloon 244 contacts a portion of graft material 242 and holds it in place for a subsequent cutting step (for example, as described herein).

While FIG. 5B depicts a deflatable/inflatable balloon 244 configured to grasp graft material 242, FIGS. 5C and 5D depict self-expanding balloon 246 (e.g., a self-expanding mesh balloon) as an alternative for grasping graft material 242. FIG. 5C is an isolated, schematic view of self-expanding balloon 246 connected to catheter 248. Self-expanding balloon 246 and catheter 248 are configured to collectively advance along guidewire 224. FIG. 5D is a schematic, side view of sheath 250 and self-expanding balloon 246 located outside of sheath 250 in a radially expanded state. Self-expanding balloon 246 is in a radially compressed state when packaged inside sheath 250. Sheath 250 may be retracted or balloon 246 advanced to change self-expanding balloon 246 from the radially compressed state to the radially expanded state. Once in the radially expanded state, self-expanding balloon 246 may be retracted by retracting catheter 248 to contact graft material 242 and hold it in place for the subsequent cutting step.

While FIGS. 5C and 5D depict needle 222 extending in a linear direction, FIGS. 5E through 5G depict schematic, side views of catheter 251 having distal coil 252 extending in a curved direction (e.g., a helical direction). FIG. 5E shows catheter 251 advancing through catheter 208 such that distal coil 252 of catheter 251 extends beyond the distal end of catheter 208. FIG. 5F is a magnified, side view of distal coil 252 showing its helical shape and including sharpened tip 254. As represented by the arrows shown in FIG. 5E, rotation of catheter 251 about its longitudinal axis causes rotational movement of distal coil 252. As shown in FIG. 5E, advancing sharpened tip 254 along the longitudinal axis of needle 222 pierces graft material 242. As shown in FIG. 5G, the rotational movement of distal coil 252 causes distal tip 252 to thread graft material 242, thereby advancing distal tip further through graft material 242 and further into a branch blood vessel. Once distal coil 252 is in this advanced position, distal coil 252 is anchored to graft material 242 such that longitudinal movement of wire 251 along its axis does not dislodge distal coil 252 from graft material 242. Instead, graft material 242 is drawn inward as catheter 251 is retracted away from peripheral artery 206.

FIGS. 5H and 5J depict an alternative grasping feature where a vacuum is applied to through lumen 216 of steerable catheter 208. FIG. 5H depicts a schematic, side view of steerable catheter 208 where distal end 256 of steerable catheter 208 contacts graft material 242. Vacuum is then applied to lumen 216 (as represented by arrow 258) to create suction between graft material 242 and steerable catheter 208. The vacuum causes a portion of graft material 242 to draw into lumen 216 of catheter 208, thereby grasping graft material 242.

One or more of the grasping features (e.g., mechanical, guidewire/balloon, coiled tip, and/or vacuum) may be used to grasp the graft material. The grasped material may be cut as further described herein. The grasping of the material may facilitate controlled cutting of graft material aligned with the peripheral or branch blood vessel. The pulling force (e.g., mechanical or vacuum pulling force) may be increased or decreased to increase or decrease the amount of graft material 242 subject to the cutting operation. FIG. 5J depicts a schematic view showing the correlation between the amount of grasped material 258 compared to the diameter of cut material 260 after the cutting operation.

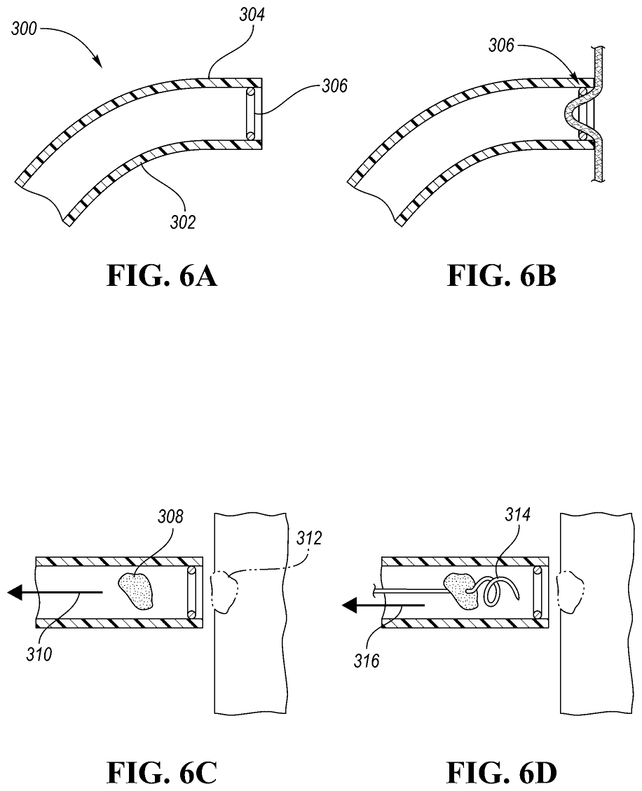

FIGS. 6A through 6D depict RF ablation device 300 configured to cut graft material of a deployed stent graft at a fenestration site. Catheter 302 includes distal region 304 having ring electrode 306 fit inside distal region 304. Locating ring electrode 306 inside distal region 304 avoids or mitigates damaging the stent graft or proximate tissue. While FIG. 6A shows ring electrode 306 offset from the distal end of catheter 302, ring electrode may flush with the distal end or closer to the distal end than shown to cut relatively more graft material at the fenestration site. FIG. 6B depicts graft material pulled inward into catheter 302 using a grasping feature (e.g., mechanical, guidewire/balloon, coiled tip, or vacuum). Ring electrode 306 is excited by RF energy to cut the graft material at ring electrode 306 to form cut graft material and a fenestration in the graft material. The inwardly pulled graft material creates a valley of graft material and ring electrode 306 cuts around a perimeter of the valley to form a substantially circular piece of cut graft material and a circular fenestration. By locating ring electrode 306 offset the distal end of catheter 304, only material pulled slightly into distal region 306 is cut, thereby avoiding, or eliminating contact with the graft frame or patient tissue during energy delivery. In one or more embodiments, RF ablation is used because it cauterizes (e.g., melts or fuses) the edges of the cut graft material and avoids or reduces frayed edges and emboli. While RF ablation may be one mechanism to create the fenestration, any other suitable mechanism may be used, such as resistive heating.

The severed graft material can be extracted via the same forces used to pull the graft material against the electrode and remove the cut graft material from the fenestration site. FIG. 6C depicts a schematic, side view of vacuum force used to extract cut graft material 308 through catheter 302 as represented by arrow 310. Fenestration 312 is formed by the cutting operation. The vacuum force may be synchronized with RF energy pulses for cutting the graft material at the fenestration site to not only remove the ablated graft material, but also any off-gassing emboli produced by the process. FIG. 6D depicts a schematic, side view of helical tip 314 used to extract cut graft material 308 though catheter 302 as represented by arrow 316. While a mechanical device is disclosed in FIG. 6D to grasp and remove the cut graft material, a vacuum source may be additionally used to aspirate any emboli produced. While FIG. 6D discloses helical tip 314 as the mechanical device, other examples of non-limited mechanical devices suitable for grasping and removal of cut graft material include balloons and mesh.

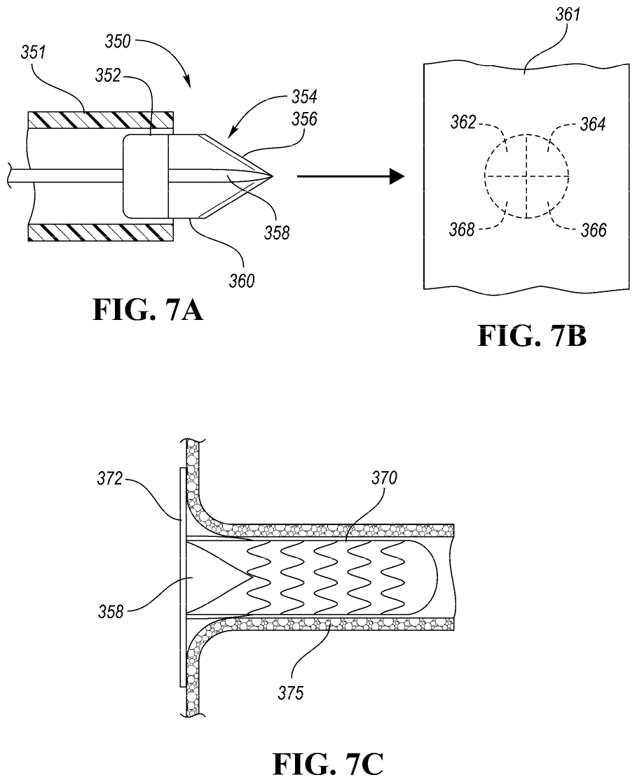

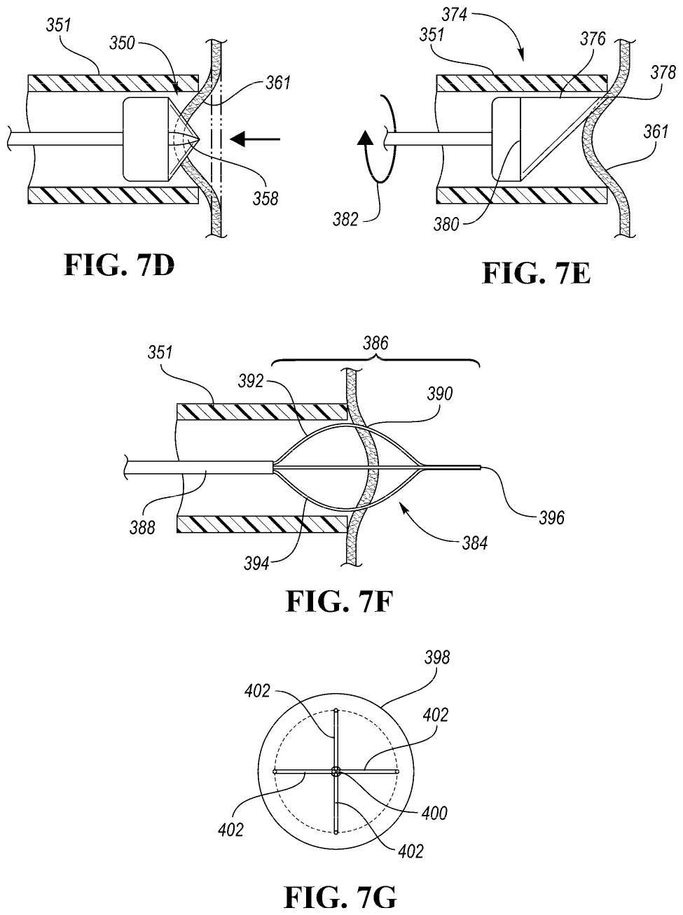

FIGS. 7A through 7G depict mechanical cutting devices configured to cut graft material at a fenestration site. In one or more embodiments, these mechanical cutting devices may be used an alternative to RF energy delivery (or other non-mechanical cutting methods). As a benefit of one or more of these embodiments, a recessed cutter device (as described herein) avoids or reduces patient harm by reducing the likelihood of contact with patient tissue. In one or more embodiments, a vacuum created within the main catheter may assist in gripping the graft material at the fenestration site and/or aspirate any emboli produced.

FIG. 7A depicts a schematic, side view of cutter 350 including base portion 352 and blade portion 354 extending therefrom. Cutter 350 is delivered to a fenestration site through catheter 351 (e.g., a steerable catheter). Base portion 352 includes a smooth peripheral edge to center cutter 350 as it advances through catheter 351 (or is retracted therefrom). As shown in FIG. 7A, the smooth peripheral edge is circular. The circular smooth peripheral edge may extend beyond the circumference of the cutting blades to mitigate or eliminate contact between the cutting blades and the inner surface of catheter 351 as cutter 350 travels through catheter 351. Blade portion 354 includes first blade 356, second blade 358, third blade 360, and fourth blade (not shown). Adjacent blades are radially offset each other by ninety degrees, but other angular offsets are contemplated. The blades taper inward from base portion 352 toward the distal tip of blade portion 354 to help facilitate cutting into graft material 361.

FIG. 7B depicts a plan view of graft material 361 cut by blade portion 354 of cutter 350. As shown in FIG. 7B, blade portion 354, when advanced into the graft material, cuts first, second, third, and fourth flaps 362, 364, 366, and 368 of graft material 361 configured to rotate outward about a perimeter portion to form an opening into a peripheral or branch blood vessel. While FIGS. 7A and 7B depict four (4) blades cutting four (4) flaps, in other embodiments, 3, 5, 6, 7, 8, or more blades can be used to create 3, 5, 6, 7, 8, or more flaps.

FIG. 7C shows a schematic, side view of branch stent graft 370 deployed in the opening formed by first, second, third, and fourth flaps 362, 364, 366, and 368. Branch stent graft 370 opens into main stent graft 372, which includes graft material 361. Branch stent graft 370 pushes first, second, third, and fourth flaps 362, 364, 366, and 368 against the walls of blood vessel 375 to form a tight fit between branch stent graft 370 and first, second, third, and fourth flaps 362, 364, 366, and 368 to mitigate or eliminate leakage at the joint between main stent graft 372 and branch stent graft 370.

FIG. 7D depicts a schematic, side view of an alternative cutting operation where graft material 361 is forced inward catheter 351 using any mechanism disclosed herein. The cutting operation is at least partially performed within catheter 351 using cutter 350, thereby mitigating or eliminating tissue damage from the cutting operation.

FIG. 7E depicts a schematic, side view of an alternative cutting operation where graft material 361 is forced inward catheter 351 and cut using rotary cutter 374. Rotary cutter 374 includes tapered blade 376 tapering outward from distal end 378 to proximal end 380 to open blade 376 to adequate contact with inwardly situated graft material 361 for cutting the graft material 361 within catheter 351. The cutting operation is performed by rotating (as shown by arrow 382) and/or advancing blade 376 relative to catheter 351. The cutting operation is at least partially performed within catheter 351 using rotary cutter 374, thereby mitigating or eliminating tissue damage from the cutting operation.

FIG. 7F depicts a schematic, side view of wire cutter 384 including convex wire cutting elements 386 extending from inner catheter or wire 388. Wire cutter 384 is delivered to a fenestration site through catheter 351 (e.g., a steerable catheter). Convex wire cutting elements 386 include first wire 390, second wire 392, third wire 394, and fourth wire (not shown). Adjacent wires are radially offset each other by ninety degrees. The wires curve inward from a middle portion toward their proximal ends merging at inner catheter or wire 388 and distal ends of the wires merging at distal tip 396. The wires may mechanically cut through the graft material with or without additional energy being applied to the wires, such as ultrasound energy, RF energy, or other mechanisms for translating energy from a proximal end of the wires to the distal end where the cut is made.

FIG. 7G depicts a plan view of graft material 398 cut by wire cutter 384. As shown in FIG. 7G, distal tip 396 is advanced through graft material 398 at center 400 and as wire cutter 384 is further advanced, first wire 390, second wire 392, third wire 394, and fourth wire (not shown) tear through graft material outward along segments 402 to form four (4) flaps configured to rotate outward about a perimeter portion to form an opening into a peripheral or branch blood vessel. While FIGS. 7F and 7G depict four (4) wires cutting four (4) flaps, in other embodiments, 3, 5, 6, 7, 8, or more blades can be used to create 3, 5, 6, 7, 8, or more flaps.

The fenestration devices disclosed in this section may be delivered via femoral or supra-aortic access into an implanted stent graft and to a branch vessel using a steerable catheter system. In one or more embodiments, a combination of a guidewire, vacuum, and RF energy may be used to access, grip, and remove the intended graft material, respectively. The delivery system may be a steerable lumen with an internally tip housed RF ablation ring. The hollow internal lumen may be configured to allow for needle, guidewire, or other disclosed access components to de delivered to branch vessel location while also allowing aspiration to remove graft or any procedure developed emboli. The steerable system may facilitate access to multiple geometries and anatomies to treat an increased number of the patient population.

In one embodiment, femoral or supra-aortic access may be obtained, and a steerable catheter may be tracked to a branch vessel location using Fluro and/or echo guidance. Catheter flex may be applied, and the catheter tip may be oriented perpendicular to a graft wall at a desired branch vessel location. Probing may be used to look for tenting of the graft material at a branch vessel location if a bare stent marker was not previously placed. Aspiration vacuum may be applied and/or a hollow needle may be deployed to place a guidewire across graft and into the branch blood vessel. Alternatively, a helical tip mandrel is threaded through the graft material, or a balloon or mesh gripper is placed over a guidewire through the graft material to obtain access and to grip the graft material. The graft material is vacuum and/or mechanically pulled into the catheter tip to contact an RF ring, which is then energized. The RF energy ablates desired graft material while the vacuum aspirates the graft material and any additional emboli associated with the material removal into the catheter. The delivery system is subsequently removed from the anatomy.

One or more of the embodiments disclosed in this section have one or more of the following benefits. The active steering using a steerable tip catheter permits more precise fenestration positioning. One or more of the grasping devices and/or methods provide better placement and cutting precision control. As disclosed in one or more embodiments, pulling graft material into a catheter allows for forming larger fenestration hole sizes than catheter delivery, allowing for reduced crossing profile. Vacuum aspiration permits improved emboli and graft material removal, as well as enhancing the safety of using RF energy. A recessed RF electrode may avoid contacting patient tissue or graft frame material for supporting patient safety. In one or more embodiments, a recessed cutter is used to avoid contacting patient tissue or graft frame material contact.

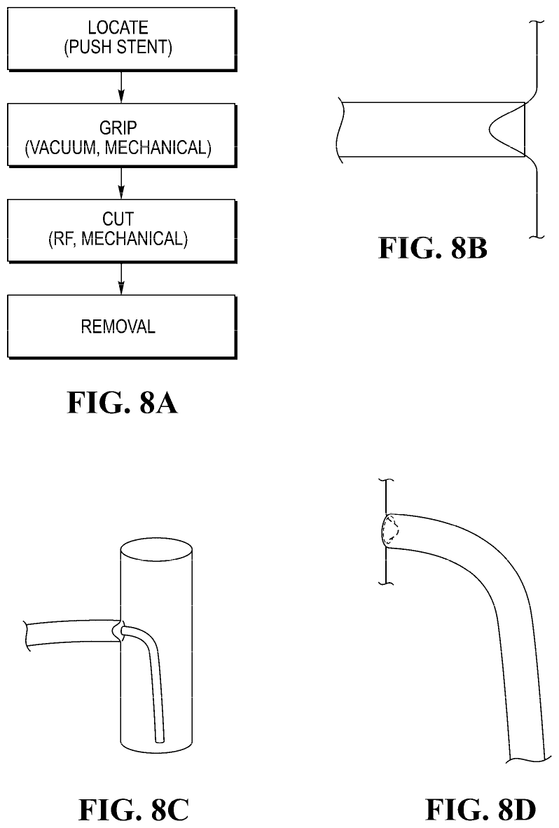

FIGS. 8A through 8D depict an embodiment directed to locating, gripping, cutting, and removing of graft material at a fenestration site.

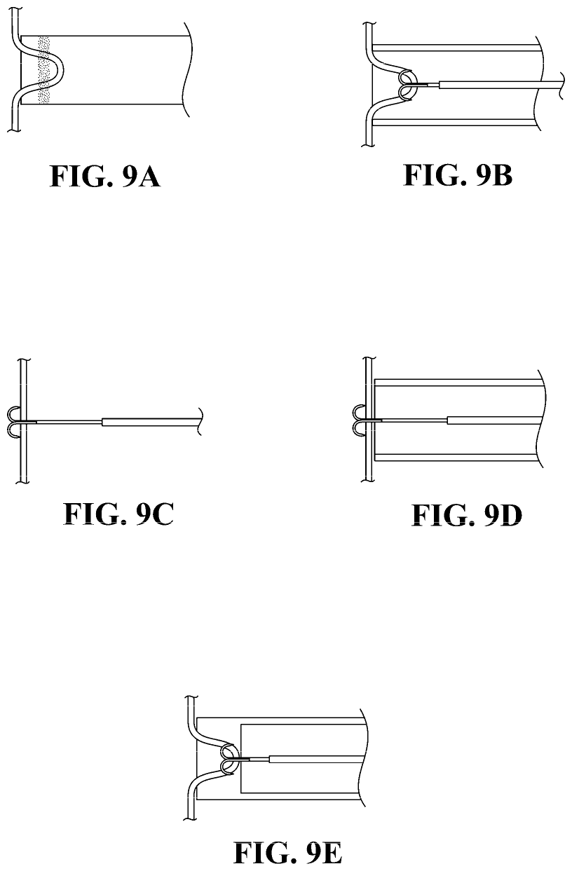

FIGS. 9A through 9E depict embodiments directed to locating, gripping, cutting, and removing of graft material at a fenestration site.

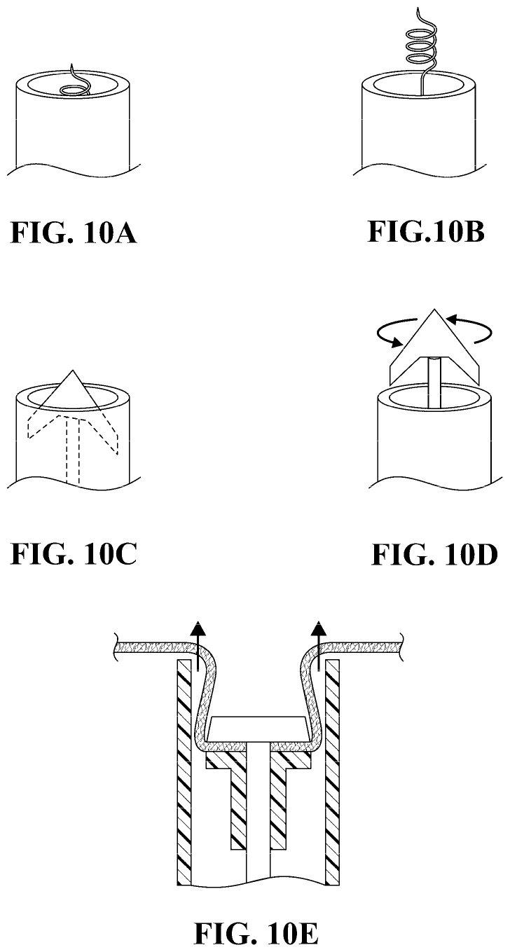

FIGS. 10A through 10E depict embodiments directed to gripping and cutting of graft material at a fenestration site.

In one or more embodiments, an in-situ fenestration device for locating a fenestration site and for forming a fenestration is disclosed. The device includes first and second magnetic locators configured to magnetically mate with each other within a patient's vasculature. One or both first and second magnetic locators may be releasable. The device may further include first and second heating elements configured to heat the first and second magnetic locators to form a fenestration at the fenestration site. The first and second heating elements may include one or both the first and second magnetic locators.

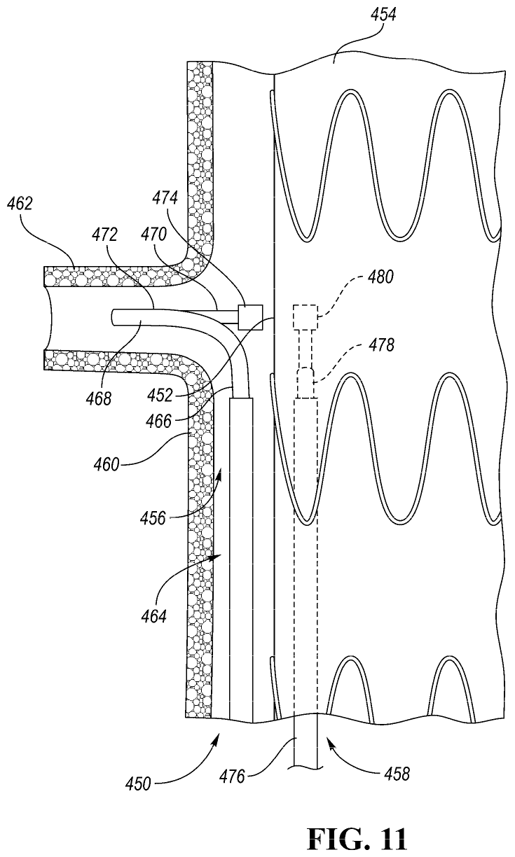

FIG. 11 depicts a schematic, side view of magnetic locator system 450 configured to locate fenestration site 452 on deployed stent graft 454. Magnetic locator system 450 may be beneficial at locating branch or peripheral blood vessels (e.g., the renal arteries) to generate fenestrations at appropriate locations to permit perfusion to the branch or peripheral blood vessels after they are covered by stent grafts. FIG. 11 depicts first magnetic locator device 456 and second magnetic locator device 458 deployed within abdominal aorta 460. Deployed stent graft 454 covers branch artery 462 extending from abdominal aorta 460. Magnetic locator system 450 may be used to locate fenestration site 452 that is later fenestrated to restore perfusion between abdominal aorta 460 and branch artery 462. The magnetic components may have a snap attraction to each other.

First magnetic locator device 456 may be placed within abdominal aorta 460 prior to deployment of stent graft 454. First magnetic locator device 456 includes sheath 464 and catheter 466 including bendable distal region 468, which may be formed of a polymeric material. Catheter 466 is configured to track through the lumen of sheath 464 to an advanced position where bendable distal region 468 extends beyond sheath 464. As shown in FIG. 11, bendable distal region 468 is in a bent position where a portion thereof extends into branch artery 462. Bendable distal region 468 includes an opening, trench, or strip (as further described below) configured to permit magnet device 470 to extend out of bendable distal region 468 in an extended position. Magnet device 470 may be fixedly connected to fixing portion 472 of bendable distal region 468. In the extended position, first magnet 474 of magnet device 470 extends toward the longitudinal axis of abdominal aorta 460 and is aligned with branch artery 462. The extended position may be achieved before or after deployment of stent graft 454.

After first magnetic locator device 456 is deployed relative branch artery 462, stent graft 454 may be deployed within abdominal aorta 460. The stent graft 454 may be partially deployed (e.g., with diameter reducing ties) or fully deployed. After partial or complete deployment of stent graft 454, second magnetic locator device 458 is deployed within the lumen of stent graft 454. Second magnetic locator device 458 includes sheath 476 and catheter 478 carrying second magnet 480 on its distal end. Catheter 478 is configured to track through the lumen of sheath 476 to an advance position where second magnet 480 extends beyond distal end of sheath 476. In one deployment scenario, after catheter 478 is deployed such that, its distal end is in the vicinity of branch artery 462, catheter 478 is tracked through the lumen of sheath 476 such that second magnet extends beyond the distal end of sheath 476 and second magnet 480 aligns with first magnet 474.

Once the first and second magnets 474 and 480 are aligned, the first and second magnets 474 and 480 are magnetically mated with fenestration site 452 therebetween. At this point, and described in more detail below, a fenestration is cut at fenestration site 452 using a cutting operation (e.g., inductive heating, cautery element, RF ablation, etc.). Thereafter, first and second magnetic locator devices 456 and 458 are removed from the patient's vasculature. Magnetic device 470 may be withdrawn into bendable distal region 468 as bendable distal region 468 is straightened and retracted into the lumen of sheath 466, thereby reducing the chance that bendable distal region 468 and/or magnetic device 470 is caught on the patient's vasculature during retraction of first magnetic locator device 456. Catheter 478 may be retracted into sheath 476 before second magnetic locator device 458 is retracted and removed from the patient's vasculature. The branch vessel and fenestration are stented (e.g., with a branch stent graft) to maintain alignment between the two and permit lasting perfusion between the main artery and the branch vessel.

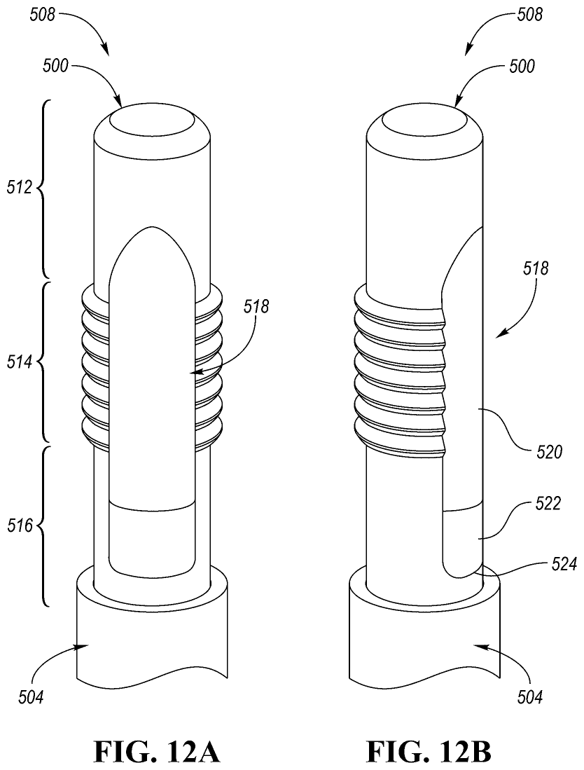

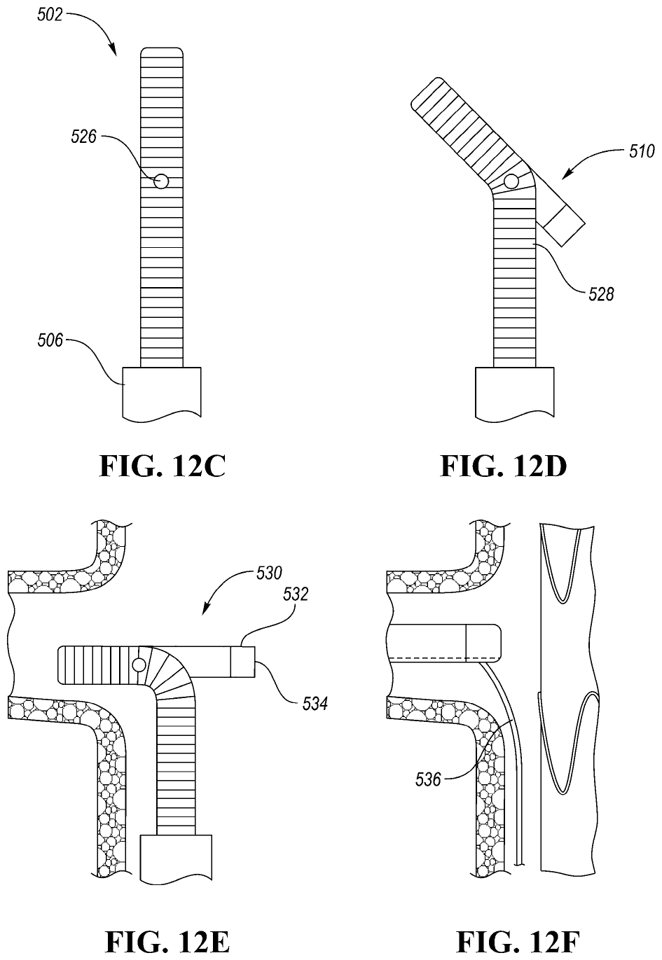

FIGS. 12A through 12F depict schematic, side views of distal bendable portions 500 and 502 of catheters 504 and 506, respectively, of magnetic locator devices 508 and 510, respectively.

FIGS. 12A and 12B show bendable distal portion 500 of catheter 504 of magnetic locator device 508. Bendable distal portion 500 includes distal end section 512, middle section 514, and proximal end section 516. Middle section 514 includes a series of bellows configured to bend bendable distal portion 500 from a straight position (as shown in FIGS. 12A and 12B) to a bent position (as shown in FIGS. 12D and 12E). The bending operation may be performed by a pushing or pulling force exerted on middle section 514. In one or more embodiments, distal end section 512 and proximal end section 516 are partially or completely rigid and do not include any bellows. Bendable distal portion 500 defines trench 518 formed therein. Trench 518 exposes magnetic device 520, which includes magnet 522 disposed on one end thereof. Front face 534 of magnet 522 faces proximal when distal bendable portion 500 is in the straight position. Front face 524 of magnet 522 aligns with a branch blood vessel and faces away from the branch blood vessel when bendable distal portion 500 is in the bent position such that a portion of distal bendable portion 500 and magnetic device 520 lie in the same longitudinal axis.

Magnet 522 (e.g., front face 534 of magnet 522) has a ferromagnetic characteristic such that it mates with an opposing magnet (e.g., magnet 480 shown in FIG. 11). The ferromagnetic characteristic may be a permanent magnet. The permanent magnet may be demagnetized with heat provided from the implement used to make a fenestration at the fenestration site. Alternatively, the ferromagnetic characteristic may be an electromagnet configured to be electrically magnetized and demagnetized. One permanent magnet may be mated with an electromagnet.

FIGS. 12C through 12E shows bendable distal portion 502 of catheter 506 of magnetic locator device 510. Bendable distal portion 502 includes pivot axis 526 such that bendable distal portion 502 is configured to change from a straight position as shown in FIG. 12C, to a first bent position as shown in FIG. 12D, and to a second bent position as shown in FIG. 12E. As shown in FIGS. 12C through 12E, the entire length of distal bendable portion 502 may be bendable. Bendable distal portion 502 defines trench 528 formed therein. Trench 528 exposes magnetic device 530, which includes magnet 532 disposed on one end thereof. Front face 534 of magnet 532 aligns with branch blood vessel 536 and faces away from branch blood vessel 536 when distal bendable portion 502 is in the bent position such that a portion of bendable distal portion 502 and magnetic device 530 lie in the same longitudinal axis.

If magnetic locator system 450 is delivered via an iliac artery, there may not be enough room to deliver first magnetic locator device 456 and second magnetic locator device/fenestration cutter 458 within the same femoral artery. In such an instance, the first locator may reduce down to a wire 536 as shown in FIG. 12F such that the second magnetic locator/hole cutter can pass through the same iliac and the stent graft is delivered through the other iliac. For example, the distal bendable portions 500 and 502 may be connected to a guide wire 536 at their proximal ends instead of being connected to a catheter. This may leave only the wire 536 in the femoral artery, allowing more space for the second locator device 458 to be tracked therethrough. The locator device connected to the wire may be tracked through the sheath 466 or a separate guide catheter within the sheath 466 that may be withdrawn after delivery. In another embodiment, first magnetic locator device 456 may be delivered using a brachial approach such that both iliac arteries are available for the stent graft main body and a second locator/hole cutter. A relatively larger diameter locater can be left in the access vessels. While a brachial approach may allow for a larger diameter first locator (e.g., attached to a catheter), the embodiment with wire 536 may also be used with a brachial approach.

A renal artery may range from 3 mm to 8 mm in diameter. A locator may range from 2 mm to 5 mm in diameter to allow for renal blood flow during delivery of the magnetic locator device.

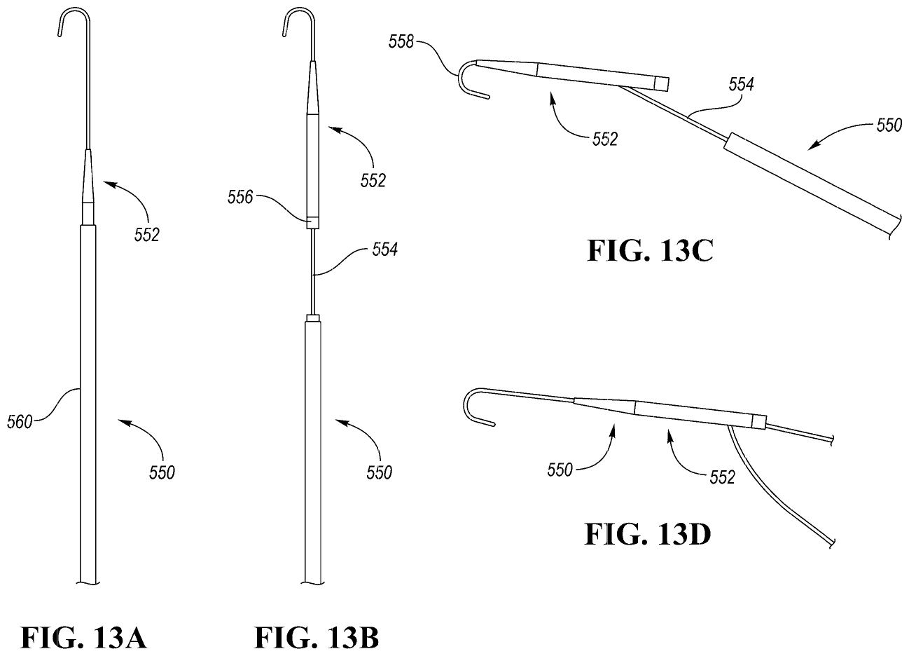

FIGS. 13A through 13D depict images of magnetic locator device 550 configured to be deployed within a patient's vasculature using the femoral/iliac arteries. Dilator 552 includes a slotted proximal region (e.g., having a c-shaped cross section) so when placed in a renal artery, guidewire 554 exits out of the slotted proximal region. Proximal end 556 of dilator 552 is formed of a magnetic material (e.g., iron or a magnet) such that the face of proximal end 556 can mate flush with another magnetic component (e.g., magnet 480 of second magnetic locator device 458). Dilator 552 may have a diameter of any of the following or in a range of any two of the following: 6, 7, 8, 9, 10, 11, 12, 13, 14, and 15 French. Guidewire 554 may have a diameter of any of the following or in a range of any two of the following: 0.018, 0.020, 0.250, 0.030, 0.035, and 0.040 inches.

Deflectable tip 558 of guidewire 554 is configured to stop dilator 552, including the magnetic component thereof, from advancing too far and becoming disengaged from guidewire 554. Dilator 552, including the magnetic component thereof, may be removed from catheter 560 once it is detached from the other magnetic component. In one or more embodiments, a wire is attached to a dilator configured to maintain control over the dilator so that it does not get lost, and another wire to track over can be used as depicted in FIG. 13D.

In an alternative embodiment, a guidewire is fixed to a dilator within a slotted side of the dilator to prevent the locator from being lost. The fixed wire exits out of the slotted side and has a pre-formed curve, so the wire does not interfere with mating magnetic components. A central wire is used for tracking and is removed during the mating of the magnetic components. In one or more embodiments, a wire fixed to the dilator has a deflectable tip or section so the wire can be curved when placed in a target vessel but then straightened again when recaptured into the catheter for removal.

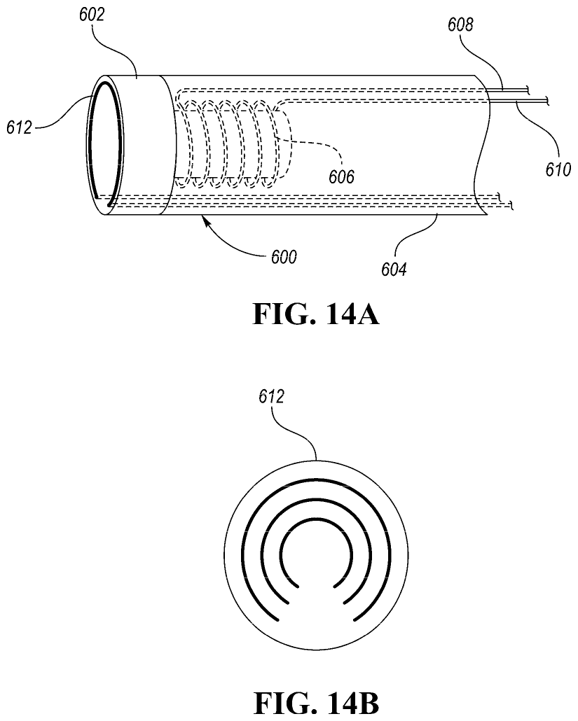

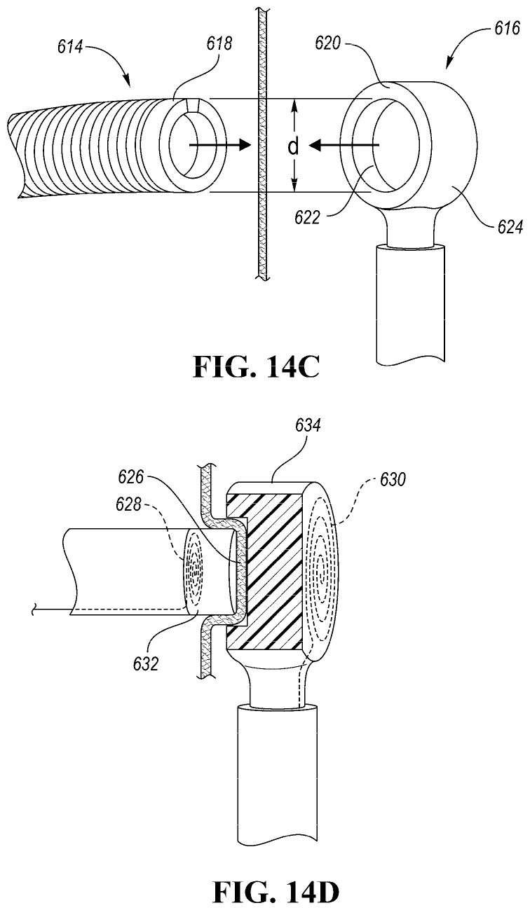

FIGS. 14A and 14B depict schematic views of embodiments of mechanisms for magnetizing magnetic locator devices. FIG. 14A depicts magnetic component 600 including head portion 602 extending beyond catheter 604 and base portion 606 extending into the lumen of catheter 604. Base portion 606 has a diameter less than the diameter of head portion 602. First electromagnetic wire 608 is electrically connected to head portion 602 and second electromagnetic wire 610 is electrically connected to base portion 606. Magnetic component 600, first electromagnetic wire 608 and second electromagnetic wire 610 collectively form a circuit such that a magnetic field may be turned on or off at face 612 of magnetic component 600. Magnetic component 600 may be formed of a permanent magnet material (e.g., neodymium). Magnetic component 600 may demagnetize with heating (as magnetic component 600 reaches its Curie temperature). As shown in FIG. 14B, face 612 of magnetic component 600 may have electrodes having different diameters (e.g., 3, 5, or 7 mm). The different diameter may optimize heat distribution in components A and B during punch-melting of a graft material in vivo. In one embodiment, as shown in FIG. 14A, for example, face 612 may a single cutting element size and the clinician selects the size prior to insertion into the patient's vasculature. In another embodiment, as shown in FIG. 14B, for example, the clinician, after insertion, may selectively engage (e.g., energize) whichever cutting element size desired (e.g., a range of sizes are present on the heating or cutting head). A single C-shaped cut permits passage through graft material but also retains cut portion attached to graft.

FIGS. 14C and 14D depict schematic views of the mating of first and second magnetic locator devices 614 and 616. As shown in FIG. 14C, first magnetic locator device 614 includes face 618 having a face diameter and second magnetic locator device 616 includes face 620 including an indentation 622 having an indentation diameter. The outer diameter of face 618 correlates to the fenestration diameter in one or more embodiments. The face diameter is less than or equal to the indentation diameter such that a portion of first magnetic locator device 614 resides within indentation 622 when the first and second magnetic locator devices 614 and 620 are magnetized. The side surface of indentation 622 is configured to aid in fixing the location of face 620 within indentation 622. Indentation 622 terminates at side-facing surface 624, which is configured to be magnetized.

As shown in FIG. 14D, stent graft material 626 is located within indentation 622 between face 620 and side-facing surface 624 when the first and second magnetic locator devices 614 and 620 are magnetized. The side surface of indentation 622 is configured to aid in fixing the location of face 620 within indentation 622. As shown in FIG. 14D, first magnetic locator device 614 includes first induction heating coil 628 and second magnetic locator device 620 includes second induction heating coil 630. First and second induction heating coils 628 and 630 are configured to heat up magnetic components 632 and 634 to cut stent graft material 624 to form a shaped fenestration. Alternatively, a resistance element heats up magnetic components 632 and 634. Non-limiting examples of resistance elements include a bovie-knife or a cautery knife. In one or more embodiments, the electromagnetic/induction heating coils may be less than or equal to 8 mm (25 Fr) to track through using the femoral/iliac approach.

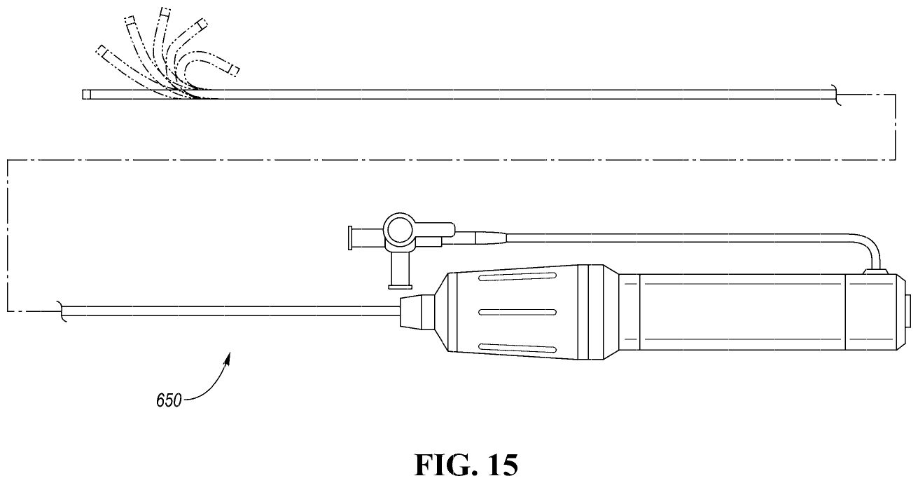

FIG. 15 depicts a side view of steerable catheter device 650. A hole cutter mechanism (e.g., first and second magnetic devices) may be included on a tip of steerable catheter device 650 or the hole cutter mechanism may be normal (e.g., 90 degrees) relative to the catheter axis. Non-limiting examples of steerable catheters suitable for use with one or more embodiments used herein include the Aptus system available from Medtronic PLC of Minneapolis, Minnesota. The steerable catheter may be actuated via a pull wire. The catheter can be configured to steer to snap with a magnetic locator in a side branch.

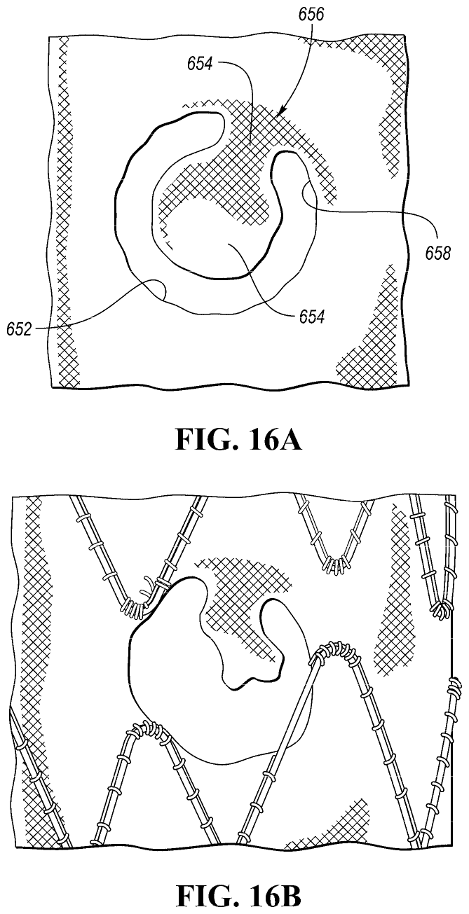

FIGS. 16A and 16B depict schematic views of holes formed using a magnetic locator system of one or more embodiments. As shown in FIG. 14C, first magnetic locator device 614 includes face 618 having a c-shaped profile. Face 618 is configured to form c-shaped aperture 652 (e.g., a horseshoe-shaped cut) and flap 654 in stent graft material 656. Edges 658 of c-shaped aperture 652 can be melted by the magnetic locator system to strengthen edges 658. In another embodiment, the magnetic locator system may form a continuous aperture (e.g., with an O-shaped) and cut material therein. In one or more embodiments, a magnetic sandwich allows extraction of the cut material if one of the magnetic locators is releasable. Non-limiting examples of cutters include an electrocautery element, inductive heating, RF ablation, or ceramic blades.

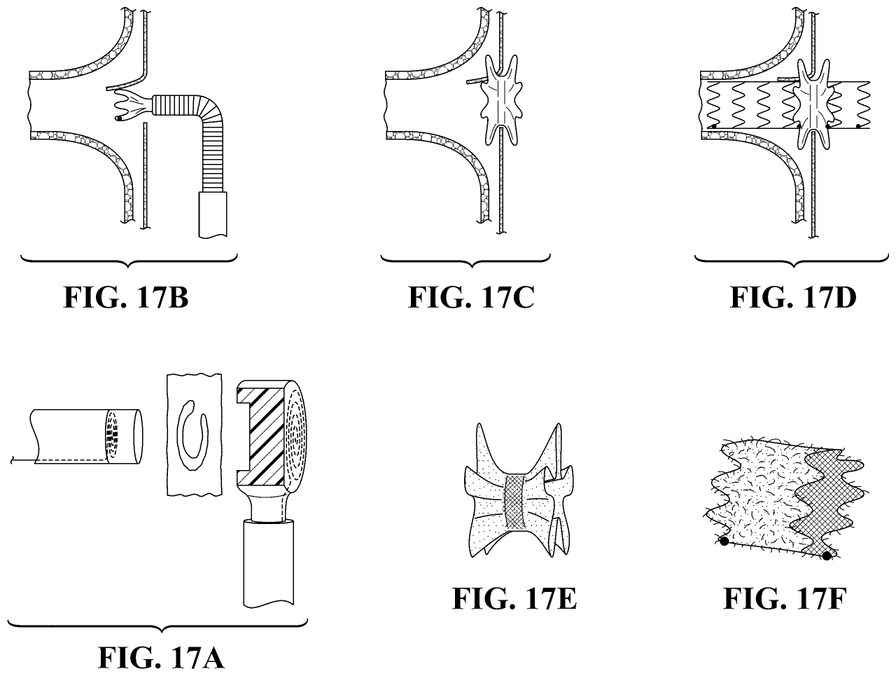

FIGS. 17A through 17F depict schematic views of alternative embodiments of grommet stents/stent grafts and expanding flanges on either side of the graft wall to strengthen the edge of the aperture formed by the magnetic locator system.

Following are procedural steps according to one or more embodiments. Before inserting a stent graft, (1) each renal is cannulated with an angiographic catheter and wire, (2) the angio catheters are removed leaving a wire in each renal, (3) a locator dilator is advanced within a guide catheter into each renal, and (4) the guide catheters and original wires are removed, leaving locator dilators and support wires in each renal. The stent graft may be partially inserted up the ipsilateral artery. The stent graft may be partially deployed (e.g., with diameter reducing ties) or fully deployed. A steerable catheter (e.g., an Aptus-like steerable catheter) is inserted within the electromagnetic tip contralateral side. By activating the electromagnetic tip, the locator dilator may be connected through the graft material. An electrode (e.g., a c-shaped electrode) may be activated to cut the graft material. At this point, the electrode and electromagnetic tip may be deactivated to release locator dilator. These steps may be repeated for cutting hole in other renal. At this point, the steerable catheter system is removed. The holes and renals may be cannulated with wires and angio catheters. The locator dilators may be removed from the body by recapturing with a guide catheter. In each renal, the angio catheters can be exchanged for guide catheters. A stent graft is fully deployed if previously partially expanded, and the guide catheters are configured to guide the holes to the renal arteries. Stents are then deployed in each hole, with flares/rivet ends with balloons, for example.

One or more embodiments may have one or more of the following benefits. The magnetic locator system of one or more embodiments may provide releasable magnets for proving reliable locations of side branch vessels (e.g., the renal arteries). The magnetic locator system of one or more embodiments may permit cutting holes in-situ directly between sandwiched magnets (e.g., providing vessel protection). Certain embodiments with only support wires remaining in the iliac are configured to allow for the introduction of cutting tools up same iliac due to lower profile.

In one or more embodiments, an in-situ fenestration device for locating a fenestration site and for forming a fenestration is disclosed. The device includes first and second magnetic locators configured to magnetically mate with each other within a patient's vasculature. The first and second magnetic locators may have complimentary curved shapes to aid in aligning the first and second magnetic locators. The device may further include first and second heating elements configured to heat the first and second magnetic locators to form a fenestration at the fenestration site. The first and second heating elements may include one or both the first and second magnetic locators.

In one or more embodiments, a method is disclosed for making an antegrade fenestration aligned with a target vessel ostium. The disclosed method includes one or more of the following benefits. Accurate positioning of the fenestration reduces or prevents axial and/or rotational misalignments that can complicate target vessel cannulation. The disclosed methods may result in fewer technical failures that require rescue interventions. The disclosed methods may reduce procedure time, x-ray and/or contrast exposures and the costs relating thereto. The disclosed methods may also reduce the likelihood of inadvertent damage to aortic tissues.

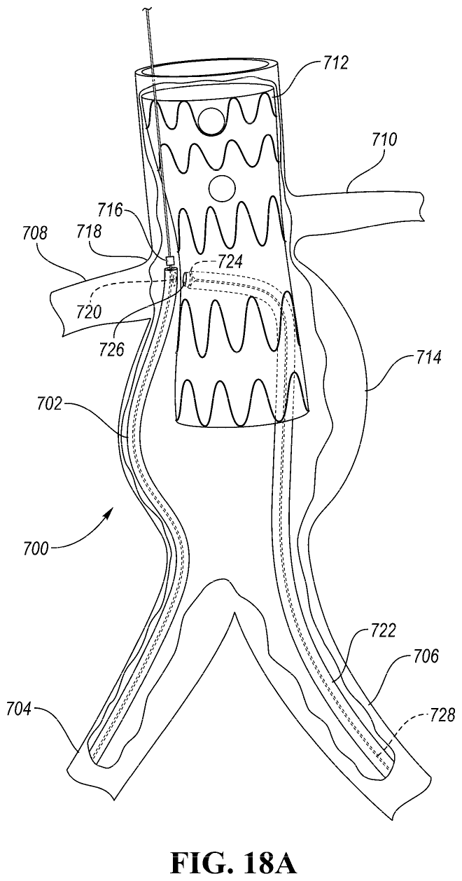

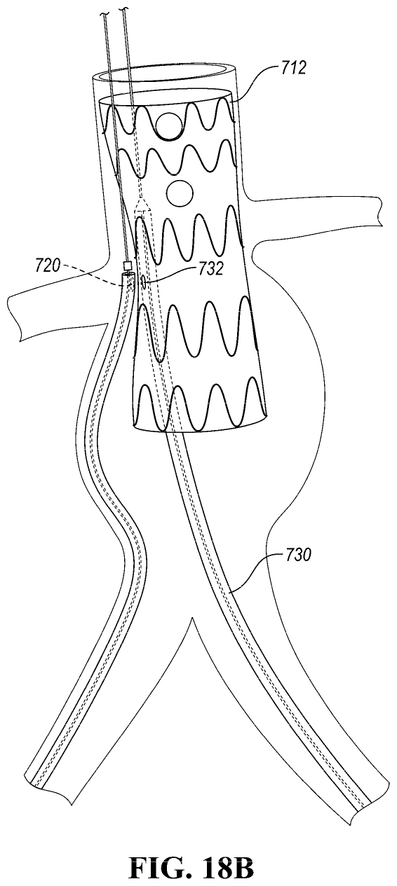

In one or more embodiments, an intravenous ultrasound (IVUS) catheter is advanced to an ipsilateral access site. FIG. 18A depicts a schematic side view of abdominal aorta 700 and the use of locator catheter 702 to locate a fenestration site. Right and left iliac arteries 704 and 706 extend from abdominal aorta 700. Right and left renal arteries 708 and 710 extend from abdominal aorta 700. Stent graft 712 in a radially expanded state is situated within abdominal aorta 700 to treat aneurysm 714. As shown in FIG. 18A, locator catheter 702 is advanced to an ipsilateral access site within abdominal aorta 700. An IVUS device may be located on a separate catheter or wire tracked through locator catheter 702. Locator catheter may have a magnet and possibly heating element components on it. The separate catheter includes IVUS transducer 716 situated on distal end thereof. Transducer 716 is configured to identify ostia of a target vessel (e.g., ostium 718 of right renal artery 708 as shown in FIG. 18A). Magnetic component 720 is attached to or integrated into the outer surface of locator catheter 702 at a distal region thereof. Magnetic component 720 may be magnetic or ferromagnetic. Magnetic component 720 may have a curved profile that follows the contour of the outer surface of locator catheter 702. During the location step, magnetic component 720 is oriented facing away from the target vessel ostium via rotation of catheter 702. In another embodiment, a locator catheter with integrated IVUS technology may be constructed to locate a target vessel ostium such that a single catheter has both imaging and magnetic and/or heating elements on it. IVUS imaging (e.g., directional or rotational) may also be used.

After locator catheter 702 is situated at the ostium of the target vessel, stent graft 712 can be partially or completely deployed at the site of aneurysm 714. The partial deployment may be executed in a staged approach using diameter reducing ties and a trigger wire.

After stent graft 712 is partially or completely deployed, fenestration catheter 722 can be introduced through the ipsilateral access site and tracked into the previously deployed stent graft 712. Fenestration catheter 722 may be a steerable catheter or a deflectable catheter configured to align distal face 724 (e.g., orthogonal alignment) with the graft material of stent graft 712. Magnetic component 726 may be disposed on distal face 724 of fenestration catheter 722. Magnetic component 726 may be oriented to align with magnetic component 720 of locator catheter 702. Magnetic component 726 may have a curved surface (e.g., concave surface) that follows the curved surface (e.g., convex surface) of magnetic component 720 to maximize the portions of the components that interact with each other. A thermocouple may also be built into the distal end of fenestration catheter 722. The thermocouple is in electronic communication with wire 728 configured to provide power to the thermocouple. As an optional safety feature, the power discharge from the thermocouple used to generate the fenestration is not enabled unless magnetic components 726 and 728 are magnetically interacting with each other. Once magnetic components 726 and 728 are magnetically interacting with each other, the fenestration can be made using the thermocouple.

FIG. 18B depicts a schematic side view of an alternative use of a magnetized thermocouple to locate a fenestration site and to form a fenestration. Fenestration catheter 730 includes magnetic component 732 formed on the outer surface of fenestration catheter 730. Magnetic component 732 may have a curved surface (e.g., concave surface) that follows the curved surface (e.g., convex surface) of magnetic component 720 to maximize the portions of the components that interact with each other. Fenestration catheter 730 may, in one embodiment, be the same catheter used to deliver stent graft 712 to the target deployment site.

Fenestration catheter 730 and/or locator catheter 702 may be oriented in-situ so that magnetic components 720 and 732 are brought into proximity to interact with each other. In one or more embodiments, one or more radiopaque (RO) markers may be place on each catheter to aid in alignment of the catheters, and the magnetized elements on each catheter may also additionally aid in the alignment process.

While FIGS. 18A and 18B depict thermal energy as a power source to create a fenestration, alternative embodiments include radiofrequency (RF) energy, laser ablation, and/or needle fenestration.

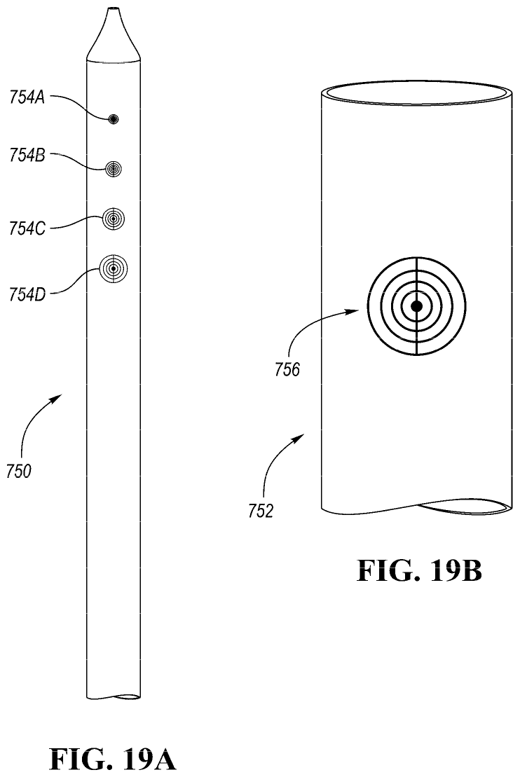

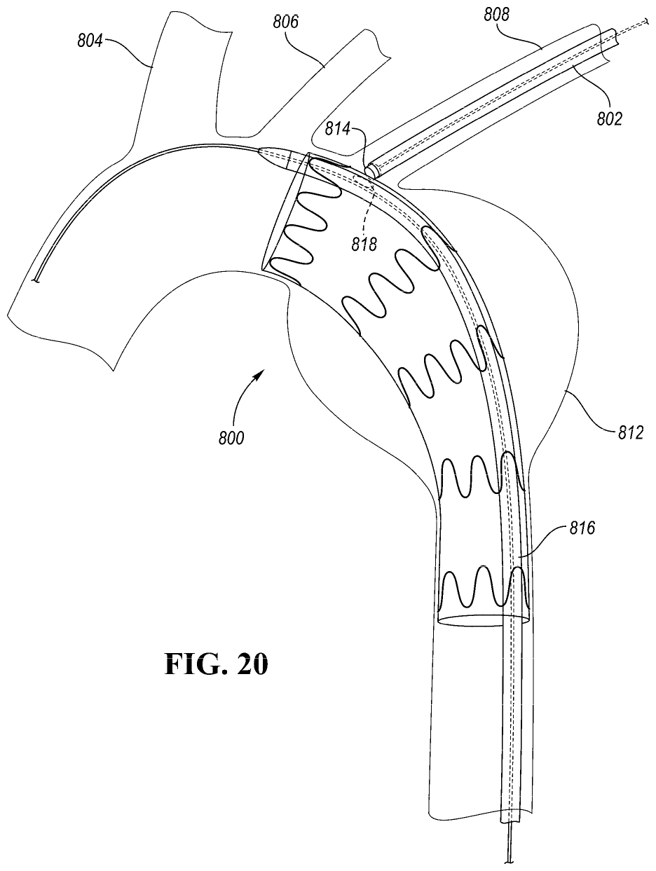

FIGS. 19A and 19B depict schematic, side views of a mechanism to size a fenestration using variably sized magnetized thermocouples. Catheters 750 and 752 may be an IVUS catheter or a fenestration catheter. Catheter 750 includes a series of magnetic components 754A, 754B, 754C, and 754D (e.g., magnetized thermocouples) increasing in size (e.g., diameter) from a distal position along the outer surface of catheter 750 toward a proximal position thereof. In other embodiments, the series of magnetic components may decrease in size (e.g., diameter) from a distal position to a proximal position. The increase or decrease in diameter may be constant. The size of the components may correspond to the size of the created fenestration. A control feature at the hub of catheter 750 can be configured to select the magnetic component from the series of magnetic components configured for a thermal discharge. The magnetic component may be selected in response to the patient's specific anatomy, the size of the branch stent graft to be inserted, or other consideration.

Catheter 752 includes a series of concentric thermal elements 756 (e.g., magnetized thermocouples) used to create a variety of fenestration sizes at a single location. As shown on FIG. 19B, thermal elements 756 are nested in a bullseye configuration. A user interface at the hub of catheter 752 can be used to determine the size (e.g., diameter) of the thermal discharge by energizing only certain elements of the thermal elements 756. The size (e.g., diameter) of selected magnetic component 754A, 754B, 754C, and 754D or selected thermal elements 756 may be selected to provide a targeted overall size for the fenestration.

FIG. 20 depicts a schematic side view of aortic arch 800 and the use of small profile catheter 802 to locate a fenestration site therein. Aortic arch 800 typically branches into brachiocephalic artery 804, left common carotid artery 806, and left subclavian artery 808. Stent graft 810 in a radially expanded state is situated within aortic arch 800 to treat aneurysm 812. Small profile catheter 802 is advanced through left subclavian artery 808 (in this embodiment).

In the aortic arch 804 where retrograde access is feasible, small profile catheter 802 with magnetic head 814 can be introduced via supra-aortic access. Catheter 802 is configured to provide a marker to allow for accurate placement of a fenestration. Catheter 802 may have a centering feature configured to locate magnetic head 814 in the center or close to the center of a target vessel ostium (e.g., balloon or wire mesh). One advantage of using a small profile catheter 802 with retrograde access is that it allows for the use of a catheter with a diameter smaller than the fenestration size to be created. Catheter 802 may be used to orient a fenestration catheter (described below) to create a large fenestration, but the small profile of the catheter 802 reduces stroke risk associated with the procedure by having less interaction with the wall of the vessel.

Fenestration catheter 816 with magnetic component 818 (as described previously) can be introduced via transfemoral access and advanced to the location of a target vessel as shown in FIG. 20. Small profile catheter 802 and fenestration catheter 816 may be aligned by torquing one or both catheters until the magnetic components interact. Subsequently, a thermal energy discharge can then be used to create a fenestration. Since femoral access allows for larger profile catheters and has reduced associated stroke risk, the fenestration catheter 816 and magnetic component 818 may have a larger diameter than catheter 802 and can create a fenestration large enough to perfuse the great vessels (e.g., up to about 18 mm) with little or no post-dilation of the fenestration.

The detailed description set forth herein includes several embodiments where each of the embodiments includes several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, the energy source/type used to create a fenestration in one embodiment may be used in any other embodiment, as well as any component or mechanism to grasp or engage graft material to be removed (e.g., vacuum/aspiration, coils, balloons, etc.). Similarly, locating features in any one embodiment (e.g., IVUS) may be incorporated into any other embodiment to facilitate location of a vessel ostium and subsequent fenestration creation at the ostium.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An in-situ fenestration device for locating a fenestration site and for forming a fenestration, the in-situ fenestration device comprising:
first and second magnetic locators configured to magnetically mate in a mated position within a vasculature of a patient at the fenestration site; and
first and second heating elements configured to heat the first and second magnetic locators to form the fenestration at the fenestration site.

2. The in-situ fenestration device of claim 1, wherein the first and second heating elements are configured to demagnetize the first and second magnetic locators to change from the mated position to a released position.

3. The in-situ fenestration device of claim 1, wherein the first magnetic locator is carried on a magnetic device configured to rotate from a delivery position to a deployment position where the first magnetic locator is facing the second magnetic locator.

4. The in-situ fenestration device of claim 1, wherein the first magnetic locator includes first and second sized heating elements having first and second sizes.

5. The in-situ fenestration device of claim 1, wherein the first magnetic locator includes an end face and the second magnetic locator includes an indentation complementary in shape to the end face.

6. The in-situ fenestration device of claim 1, wherein the first and second magnetic locators are first and second permanent magnetic locators configured to demagnetize from heat generated by the first and second heating elements, respectively.

7. An in-situ fenestration device for locating a fenestration site and for forming a fenestration, the in-situ fenestration device comprising:
a catheter having a bendable distal portion, a trench, and a magnetic device carrying a first magnetic locator, the bendable distal portion configured to bend such that the magnetic device transitions from a delivery position to a deployment position through the trench and about a pivot axis;
a second magnetic locator, the first and second magnetic locators configured to magnetically mate in a mated position within a vasculature of a patient at the fenestration site; and
first and second heating elements configured to heat the first and second magnetic locators to form the fenestration at the fenestration site.

8. The in-situ fenestration device of claim 7, wherein the first and second magnetic locators face each other when the magnetic device is in the deployment position.

9. The in-situ fenestration device of claim 7, wherein the magnetic device is fixedly connected to a fixing portion of the bendable distal portion.

10. The in-situ fenestration device of claim 7 further comprising first magnetic locator device including the magnetic device, a first sheath including the bendable distal portion, and a first catheter configured to track through the first sheath to an advanced position where the bendable distal portion extends beyond the first sheath.

11. The in-situ fenestration device of claim 7 further comprising second magnetic locator device including the second magnetic locator, a second sheath, and a second catheter configured to track through the second sheath to an advanced position where the second magnetic locator extends beyond the second sheath.

12. The in-situ fenestration device of claim 7, wherein the bendable distal portion includes bellows configured to bend bendable distal portion from the delivery position to the deployment position.

13. The in-situ fenestration device of claim 12, wherein the bendable distal portion includes a distal end section, a middle section including the bellows, and a proximal end section, and the distal and/or proximal end sections do not include the bellows.

14. The in-situ fenestration device of claim 12, wherein the trench terminates the bellows.

15. An in-situ fenestration device for locating a fenestration site and for forming a fenestration, the in-situ fenestration device comprising:
first and second magnetic locators configured to magnetically mate in a mated position within a vasculature of a patient at the fenestration site, the first and second magnetic locators having first and second complimentary shapes, respectively, configured to align the first and second magnetic locators to magnetically mate in the mated position; and
first and second heating elements configured to heat the first and second magnetic locators to form the fenestration at the fenestration site.

16. The in-situ fenestration device of claim 15, wherein the first and second complimentary shapes are first and second complimentary curved shapes, respectively.

17. The in-situ fenestration device of claim 16, wherein the first complimentary curved shape includes a convex surface and the second complimentary curved shape includes a concave surface.

18. The in-situ fenestration device of claim 15, wherein the first heating elements include first and second sized heating elements and the second heating elements include third and fourth sized heating elements, the first and third sized heating elements having a first size, the second and fourth sized heating elements having a second size.

19. The in-situ fenestration device of claim 18 further comprising a control feature to energize the first and third sized heating elements to heat the fenestration site to form the fenestration at the first size or to energize the second and fourth sized heating elements to heat the fenestration site to form the fenestration at the second size.

20. The in-situ fenestration device of claim 18, wherein the first and second sized heating elements are first concentric heating elements the third and fourth sized heating elements are second concentric heating elements.
